(19) United States
(12) Patent Application Publication
(10) Pub. No.: US 2024/0248074 A1
(43) Pub. Date: Jul. 25, 2024

(54) METHOD AND SYSTEM FOR SINGLE-CELL BIOPHYSICAL PROFILING USING A MICROFLUIDIC DEVICE

(71) Applicant: NANYANG TECHNOLOGICAL UNIVERSITY, Singapore (SG)

(72) Inventors: Han Wei HOU, Singapore (SG); Chayakorn PETCHAKUP, Singapore (SG); Haoning YANG, Singapore (SG); Linwei HE, Singapore (SG); King Ho Holden LI, Singapore (SG)

(21) Appl. No.: 18/564,484

(22) PCT Filed: May 27, 2022
(86) PCT No.: PCT/SG2022/050362

§ 371 Date: Nov. 27, 2023

(30) Foreign Application Priority Data
May 28, 2021 (SG) 10202105682R

(51) Int. Cl. G01N 33/487; G01N 15/10; G01N 15/12

(52) U.S. Cl. CPC G01N 33/48728; G01N 15/132; G01N 2015/1006; G01N 2015/133

(57) ABSTRACT

An optics-free method for single cell profiling, particularly white blood cells such as neutrophils is disclosed. Embodiments relate to multi-parametric biophysical profiling of neutrophils using a microfluidic impedance-deformability cytometry device. The device comprises a first flow channel comprising a particle focusing region, a detection region comprising a first pair of electrodes, a cell deformation zone and a second pair of electrodes. A multi-frequency impedance response of each cell before and after deformation is measured respectively by the first and second pairs of electrodes, such as to quantify membrane opacity, nucleus opacity, cell size and deformability index. A second flow channel bifurcated into two split flow pathways that converge in an intersecting manner with the first flow channel forms a cross-junction in a cell deformation zone, to hydrodynamically deform each cell by perfusing a sheath fluid along the two split flow paths from two opposite sides of the sample flow channel.

### CROSS-REFERENCE TO RELATED APPLICATION

This application claims the benefit of priority of Singapore Patent Application No. 10202105682R filed 28 May 2021, the content of which being hereby incorporated by reference in its entirety for all purposes.

### TECHNICAL FIELD

Various embodiments relate generally to an optics-free method, an optics-free system and a microfluidic device for single-cell profiling. Various embodiments are generally in the field of cell profiling using a label-free and optics-free microfluidic method. For example, various embodiments relate to multi-parametric biophysical profiling of cells (e.g. neutrophils) in a sample by using microfluidic device based on impedance cytometry.

### BACKGROUND

Neutrophils are the most abundant and key effector cells of the innate immunity¹. To resolve inflammation or eradicate pathogenic threats, they contribute to various host-defence mechanisms namely phagocytosis, chemotaxis and formation of neutrophil extracellular traps (NETs). Dysfunctions of neutrophil activities have been implicated in various diseases such as cancer^{2,3}, metabolic diseases⁴ as well as cardiovascular diseases⁵. Thus, qualitative and quantitative detection of neutrophil phenotypes and functions can be clinically critical to reveal host immunity status which can be potentially exploited for development of novel diagnosis strategies.

Typically, conventional neutrophil studies include measuring surface markers expression by flow cytometry¹⁷, or secreted cytokines using enzyme linked immunosorbent assay (ELISA)¹⁸. However, these approaches are expensive and laborious due to antibodies usage. Electrical-based microtechnologies (e.g. dielectrophoresis (DEP), electrorotation, impedance cytometry) are alternatives to facilitate label-free measurement of single cells with minimal sample requirement and cell perturbation^{9,19-21} for cell identification as well as immune cell activation¹⁰⁻¹². Recent works include leukocyte profiling using electrorotation^{22,23} and differential impedance cytometer^{10,24-26}. To enhance phenotyping capability of impedance cytometry, constricted detection channels (smaller than cell size) were used to deform cells¹³⁻¹⁶ followed by electrically quantifying entry time and transit times of single cells traversing through the channel based on detecting cell impedance to start/stop measurement of time, whereby the entry time and transit times of single cells are used to measure cell deformability. However, these techniques suffered from low throughput, being prone to clogging issues, and inaccuracies between the electrical measurements during cell squeezing and non-squeezed/native state. In contrast, while non-constriction deformability measurement methods utilizing shear flow-induced cell deformation using high flow rates or viscoelastic fluids have been developed²⁷⁻²⁹, these approaches quantified morphological changes in particle/cell shape reliant upon expensive high-speed camera (>10000 fps) and sophisticated optical setups, as opposed to being an optic-free approach. In this regard, such conventional non-constriction based techniques necessitate the use of optical quantification to analyse cell deformability for cell profiling.

Therefore, there is still need in the art for a further improved method for cell profiling, in particular, neutrophil profiling to electrically quantify biophysical properties of single cells and alleviate the abovementioned problems, such as those typically found in constriction-based deformability cytometers.

### SUMMARY

Various embodiments meet this need by providing an optics-free method for cell profiling using a specifically developed microfluidic device.

In a first aspect, there is provided an optics-free method for single-cell profiling, including:
(i) forming a single stream of cells in a sample flow channel of a microfluidic device by perfusing a fluid sample containing the cells through a particle focusing region of the microfluidic device, wherein the particle focusing region is upstream of a detection region of the microfluidic device, wherein the sample flow channel extends through the particle focusing region and the detection region;
(ii) measuring an impedance response of each cell by a first pair of electrodes disposed along the sample flow channel within the detection region;
(iii) deforming each cell within a cell deformation zone along the sample flow channel within the detection region, wherein the cell deformation zone is downstream of the first pair of electrodes;
(iv) measuring an impedance response of each cell after deformation by a second pair of electrodes disposed along the sample flow channel within the detection region of the microfluidic device, wherein the second pair of electrodes is downstream of the cell deformation zone;
(v) determining one or more quantitative relationships among impedance responses from one or a combination of the first and second pair of electrodes to quantify one or more biophysical properties of each cell for profiling of said cell in said fluid sample.

In various embodiments of such a method, the cells are blood cells, preferably myelocytes and/or lymphocytes. In specific embodiments, the cells are neutrophils. The method may thus be a method for activated neutrophil profiling.

In various embodiments of such a method, the one or more biophysical properties include:
(a) membrane opacity;
(b) nucleus opacity;
(c) cell size; and
(d) deformability index.

In various embodiments of such a method, measuring the impedance response by each pair of the first and second pair of electrodes includes measuring a multi-frequency impedance response, wherein a multi-frequency excitation signal is applied by each pair of the first and second pair of electrodes for measuring the multi-frequency impedance response.

In various embodiments of such a method, the multi-frequency impedance response includes impedance signals at three or more frequencies, wherein the three or more frequencies are in a range of about 0.1 MHz to 20 MHz.

In various embodiments of such a method, the impedance signals at the three or more frequencies include a first impedance signal at a first frequency in a range of about 0.1 MHz to 0.5 MHz, a second impedance signal at a second frequency in a range of about 1 MHz to 5 MHz, and a third impedance signal at a third frequency in a range of about 5 MHz to 20 MHz.

In various embodiments of such a method, the impedance signals at the three or more frequencies include the first impedance signal at a first frequency of about 0.3 MHz, the second impedance signal at a second frequency of about 1.7 MHz, and the third impedance signal at a third frequency of about 12 MHz.

In various embodiments of such a method, determining one or more quantitative relationships among impedance responses from the first and/or second pair of electrodes includes determining a ratio of the second impedance signal at the second frequency and the first impedance signal at the first frequency to quantify a membrane opacity of each cell as one of the one or more biophysical properties for said cell.

In various embodiments of such a method, determining one or more quantitative relationships among impedance responses from the first and/or second pair of electrodes includes determining a ratio of the third impedance signal at the third frequency and the first impedance signal at the first frequency to quantify a nucleus opacity of each cell as one of the one or more biophysical properties for said cell.

In various embodiments of such a method, further including determining the first impedance signal at the first frequency of the first pair of electrodes to quantify a cell size of each cell.

In various embodiments of such a method, determining one or more quantitative relationships among impedance responses from one or a combination of the first and second pair of electrodes includes determining a ratio of the impedance response of the first pair of electrodes and the impedance response of the second pair of electrodes to quantify a deformability index of each cell as one of the one or more biophysical properties for said cell

In various embodiments of such a method, deforming each cell within the cell deformation zone includes hydrodynamically deforming said cell by perfusing a sheath fluid along two split flow paths to converge in an intersecting manner with the sample flow channel at the cell deformation zone from two opposite sides of the sample flow channel so as to apply a hydrodynamic force for deforming each cell flowing through the cell deformation zone along the sample flow channel.

In various embodiments of such a method, the two split flow paths intersect the sample flow channel to form a cross-junction.

In various embodiments of such a method, perfusing the fluid sample is at a sample flow rate and perfusing the sheath fluid is at a sheath flow rate, wherein the sheath flow rate is higher than the sample flow rate.

In various embodiments of such a method, the sample flow rate is in the range of 1 to 20 μL/min, and the sheath flow rate is in the range of 1 to 40 μL/min.

In various embodiments of such a method, the fluid sample includes a viscoelastic medium, preferably poly(ethylene oxide), in phosphate-buffered saline, and the sheath fluid consists of a viscoelastic medium, preferably poly(ethylene oxide).

In various embodiments of such a method, forming the single stream of cells in the sample flow channel includes aligning the cells into the single stream of cells along a center of the sample flow channel by viscoelastic focusing effect.

In various embodiments of such a method, aligning the cells into the single stream of cells along the center of the sample flow channel by viscoelastic focusing effect includes perfusing the fluid sample containing the cells through a winding path section of the sample flow channel forming the particle focusing region of the microfluidic device, wherein the winding path section of the sample flow channel has a pre-determined length based on a viscoelasticity of the viscoelastic medium in order to complete alignment of the cells into the single stream of cells by viscoelastic focusing effect within the winding path section.

In various embodiments of such a method, the first pair of electrodes is immediately before the cross-junction, and the second pair of electrodes is immediately after the cross-junction.

In another aspect, there is provided an optics-free system for single-cell profiling including: a microfluidic device including a sample flow channel extending through a particle focusing region and a detection region, wherein the detection region includes a cell deformation zone, a first pair of electrodes and a second pair of electrodes, wherein the first and second pair of electrodes are respectively arranged before and after the cell deformation zone; and a computing system including: a memory; and at least one processor communicatively coupled to the memory and the first and second pair of electrodes of the microfluidic device, wherein the computing system is configured to:
(i) measure an impedance response of each cell flowing past the first pair of electrodes disposed along the sample flow channel within the detection region;
(ii) measure an impedance response of each cell after deformation flowing past the second pair of electrodes disposed along the sample flow channel within the detection region; and
(iii) determine one or more quantitative relationships among impedance responses from one or a combination of the first and second pair of electrodes to quantify one or more biophysical properties of each cell for profiling of said cell.

In various embodiments of such a system, the computing system is configured to determine a ratio of the impedance response of the first pair of electrodes and the impedance response of the second pair of electrodes to quantify a deformability index of each cell as one of the one or more biophysical properties for said cell.

In various embodiments of such a system, the system further includes a lock-in amplifier and one or more transimpedance amplifiers, wherein the lock-in amplifier communicatively coupled between the computing system and the first and second pair of electrodes of the microfluidic device, wherein each pair of the first and second pair of electrodes is configured to apply a multi-frequency excitation signal to each cell, wherein the computing system is configured to measure, via the lock-in amplifier, a multi-frequency impedance response by each pair of the first and second pair of electrodes, wherein the one or more transimpedance amplifiers is connected to one electrode of each pair to convert impedance responses for the lock-in amplifier.

In various embodiments of such a system, the computing system is configured to: measure, via the lock-in amplifier, the multi-frequency impedance response including impedance signals at three or more frequencies, wherein the three or more frequencies are in a range of about 0.1 MHz to 20 MHz

In various embodiments of such a system, the impedance signals at the three or more frequencies include a first impedance signal at a first frequency in a range of about 0.1 MHz to 0.5 MHz, a second impedance signal at a second frequency in a range of about 1 MHz to 2 MHz, and a third impedance signal at a third frequency in a range of about 10 MHz to 20 MHz.

In various embodiments of such a system, the impedance signals at the three or more frequencies include the first impedance signal at a first frequency of about 0.3 MHz, the second impedance signal at a second frequency of about 1.7 MHz, and the third impedance signal at a third frequency of about 12 MHz.

In various embodiments of such a system, the computing device is configured to determine a ratio of the second impedance signal at the second frequency and the first impedance signal at the first frequency to quantify a membrane opacity of each cell as one of the one or more biophysical properties for said cell.

In various embodiments of such a system, the computing device is configured to determine a ratio of the third impedance signal at the third frequency and the first impedance signal at the first frequency to quantify a nucleus opacity of each cell as one of the one or more biophysical properties for said cell.

In various embodiments of such a system, the computing device is configured to quantify a cell size of each cell based on the first impedance signal at the first frequency of the first pair of electrodes.

In another aspect, there is provided a microfluidic device for single-cell profiling, including: a first flow channel to form a fluid pathway for allowing a fluid sample including cells to flow from a sample inlet to an outlet, wherein the first flow channel includes a particle focusing region;
a second flow channel to form a fluid pathway for allowing a sheath fluid to flow from a sheath fluid inlet to a junction, wherein the second flow channel intersects with the first flow channel to form the junction in a cell deformation zone;
two pairs of electrodes arranged adjacent to the junction, wherein the at least two pairs of electrodes and the junction define a detection region,
wherein the two pairs of electrodes include a first pair of electrodes and a second pair of electrodes, and, wherein the first pair of electrodes extend across the first flow channel upstream of the junction, and wherein the second pair of electrodes extend across the first flow channel downstream of the junction.

In various embodiments of such a microfluidic device, the second flow channel is divided into two fluid pathways that both intersect with the first flow channel at the junction, wherein the junction is a cross-junction.

In various embodiments of such a microfluidic device, the particle focusing region is upstream of the detection region, wherein the first flow channel extends through the particle focusing region and the detection region.

In various embodiments of such a microfluidic device, the detection region includes the cell deformation zone along the first flow channel, wherein the cell deformation zone is downstream of the first pair of electrodes.

In various embodiments of such a microfluidic device, the second pair of electrodes is downstream of the cell deformation zone.

In various embodiments of such a microfluidic device, the particle focusing region is positioned upstream of the detection region.

In various embodiments of such a microfluidic device, the first pair of electrodes are substantially parallel to each other, and the second pair of electrodes are substantially parallel to each other.

All embodiments described above in the context of the method and concerning the microfluidic device and impedance detector as such are similarly applicable to the system of the various embodiments.

### BRIEF DESCRIPTION OF THE DRAWINGS

Various embodiments will be better understood with reference to the detailed description when considered in conjunction with the non-limiting examples and the accompanying drawings.

FIG. 1 shows (A) a schematic of a microfluidic device according to various embodiments showing the particle focusing region 1 and detection region 2; (B) a photograph of a microfluidic device according to various embodiments in the form of a chip filled with dye for visualization; (C) overlaid brightfield image illustrating hydrodynamic cell deformation by sheath fluid at a channel cross junction (Scale bar is 20 μm) with the cell deformation zone 3 of the microfluidic device according to various embodiments; (D) a schematic illustration of the device working principle according to various embodiments; (E) a schematic diagram of the detection region and arrangement of the two pairs of electrodes at the cross-junction according to various embodiments of the microfluidic device; (F) a schematic diagram of the particle focusing region and detection region according to various embodiments of the microflusidic device; and (G) a diagram of the system according to various embodiments illustrating the interrelationship between the hardware and software components in performing the method according to various embodiments.

FIG. 2 shows examples of particle alignment in the microfluidic device according to various embodiments for different sized beads (5, 10 and 15 μm) at sample flow rate ratio ranging from 2.5-10 μL/min.

FIG. 3 shows fluorescent images of examples of flow focusing (left) and linescan after pinching (right) to represent hydrodynamical pinching at the cross junction of the microfluidic device according to various embodiments at different flow rate ratios.

FIG. 4 shows graphs of the effect of membrane properties on the electrical opacity at different frequencies. (A) membrane conductivity (Sₘₑₘ) variation illustrates that the leaky membrane leads high opacity; and (B) membrane capacitance and permittivity variation (Cmem=A*Eₘₑₘ/d, A=surface area, and d=membrane thickness) shows that increased membrane capacitance or surface area (surface morphology of cells e.g., smooth or rough (membrane ruffle, fold)) lead to low opacity.

FIG. 5 shows graphs of the effect of nucleus properties and size on the electrical opacity at different frequency. (A) nucleus envelope conductivity (Sₙₑ) variation illustrates that the leaky membrane leads low opacity; and (B) nucleus size variation shows that increased nucleus size leads to low opacity at high frequency.

FIG. 6 shows a graph of the relation between viscosity and shear rate to characterize the viscoelastic medium.

FIG. 7 shows (A) a simulated scenario for undeformed (native) particles and deformed particles; (B) an electric field simulation in a side view and cross-sectional view; Gradient intensity indicates voltage (V) (C) impedance simulation of particles with different deformability index (major/minor axis); (D) correlation of simulated electrical deformability index with image-based normalized deformability index (with deformability index of 1.2 as a reference) with optical normalized deformability index for 8 and 12 μm particles; and (E) frequency responses of impedance for medium without particle, non-deformed (1) and deformed particles (3) (left) and differential current plot (right).

FIG. 8 shows (A) a simulated scenarios for native particle (sphere, optical deformability index (major/minor axis)=1) and deformed particle (oblate, deformability index=3) of equal volumes; (B) Side view (Z-X) and cross-sectional (Z-Y) view of electric field simulation at 0.3 MHz. Gradient intensity indicates electric field strength (V/m) with electric field lines; (C) Simulated impedance signal of particles with different shapes (optical deformability indices); and (D) Correlation of simulated electrical deformability index (with deformability index of 1.2 as reference) with optical normalized deformability index for 10 and 12 μm particles. Deformed particles (oblate spheroids) and native particles (spheroids) had the same volumes.

FIG. 9 shows (A) a representative time lapse images showing deformation profiles of HL60 cells (soft) and 15 μm polystyrene beads (stiff) flowing in the detection region of the microfluidic device according to various embodiments (sheath to sample ratio is 5:1) and snapshots of HL60 in native states and deformed states at different flow rate ratio; (B) Calculation of optical deformability index; (C) Average optical deformability index from 4 different experiments for native state, deformed state as well as normalized index (deformed/native); (D) representative time-series impedance signal according to various embodiments for 15 μm beads and HL60 at different flow rate ratios; and (E) representative impedance signal according to various embodiments for 15 μm beads and HL60 at different flow rate ratios.

FIG. 10 shows (A) Deformability profiles of DMSO treated HL-60 (control), cytochalasin D (CytoD) treated HL-60, and latrunculin B (LatB) treated HL-60. The dotted line indicates electrical deformability index at 1.2; (B) Fluorescent images of HL-60 stained with Hoechst (nucleus) and F-actin (Scale bar is 10 μm); (C) Quantification of electrical deformability index of different phenotypes (n=3); (D) Flow cytometric analysis; and (E) mean fluorescent intensity for F-actin expression (n=3).

FIG. 11 shows (A) 2D-scatter plots of electrical deformability index using different concentrations of PEO in the sample and sheath fluids; and (B) quantification of average electrical deformability index at the different concentrations. The data was from single experiment and was shown as mean±S.D.

FIG. 12 shows (A) Mechanical profiling of different HL-60 phenotypes namely untreated control (ctrl), fixed by paraformaldehyde (PFA) and differentiated into neutrophil; (B) average optical deformability index; (C) average cell size from 4 different experiments for native state; (D) Heatmap for each experimental measurements; (E) Average optical deformability index; and (F) Correlation between electrical deformability index and optical deformability index, according to various embodiments.

FIG. 13 shows (A) an overview of experimental workflow according to various embodiments; (B) Representative biophysical (membrane opacity and electrical deformabiltiy) profiling of neutrophils treated with different stimulus according to various embodiments; and Quantification of changes in membrane opacity (C), electrical deformability (D), and cell size (E) at different flow rate ratios (n=5) according to various embodiments.

FIG. 14 shows neutrophil activation profiling based on CD66b and CD11 b expression.

FIG. 15 shows (A) a heatmap of biophysical characteristics of different donors (N=5 except for PMA which is 4); and (B) 2D plots of different biophysical profiles of neutrophils.

FIG. 16 shows a heatmap correlating biophysical features (left) and a conceptual workflow for multi-parametric characterization (right).

FIG. 17 shows (A) an experimental workflow according to various embodiments for neutrophil testing and various treatment conditions (Ctrl (n=6), fMLP (n=6), Glu (n=4), PMA (n=4), HS (n=4), STS (n=3)); Flow cytometric analysis of mean fluorescent intensity (MFI) (B) CD66b and (C) F-actin expression according to various embodiments; Quantification of (D) electrical size, (E) membrane opacity, (F) electrical deformability, and (G) nucleus opacity, at different flow rate ratios according to various embodiments. Data are presented as mean±S.D. *P≤0.05 and **P≤0.01 (Compared to control).

FIG. 18 shows flow cytometric analysis for live-dead staining (Propidium iodide (PI) and Annexin V).

FIG. 19 shows representative biophysical profiles of different neutrophil phenotypes (Dotted lines indicate centroid of the control).

FIG. 20 shows the correlation between CD66b expression and biophysical features, (A) Electrical deformability index, (B) Electrical size, (C) Membrane opacity and (D) Nucleus opacity according to various embodiments.

FIG. 21 shows UMAP analysis of neutrophil biophysical features according to various embodiments: (A) Heatmap of average biophysical characteristics of different donors. The gradient bar indicates feature magnitude; (B) Localized density scatter of UMAP projection from 10 biophysical features for different neutrophil phenotypes (control, fMLP, Glucose, PMA, HS and STS). Black dots correspond to total single-cell events (n=34353) from 6 different individuals. The gradient bar indicates normalized density of the data points; (C) Magnitude maps for 4 main biophysical features namely 1) electrical size_{native, 0.3 MHz} 2) Membrane opacity, 3) Nucleus opacity, and 4) Electrical deformability index_{0.3 MHz}. The gradient bar indicates normalized magnitudes of feature.

FIG. 22 shows biophysical profiles of different individuals according to various embodiments: (A) 2D-scatter plots of membrane opacity (top), nucleus opacity (middle) and deformability index (bottom) versus electrical size; (B) Heatmap showing correlation of biophysical features where blue and red indicates positive and negative correlation respectively.

FIG. 23 shows magnitude maps (A-J) of UMAP analysis for biophysical features according to various embodiments. The gradient bar indicates magnitude intensity.

FIG. 24 shows biophysical profile of healthy control and COVID recovered patients' neutrophil: (A) Neutrophil deformability plot for heathy control (left) and COVID recovered patient (right). Dotted line indicates electrical deformability at 1; (B) 3D-scatter plot shows different biophysical parameters (electrical size, electrical deformability index, opacity); and (C) Quantification of electrical size (left, n=3), electrical deformability index (middle, n=3), opacity (right, n=3) according to various embodiments.

FIG. 25 shows a 2D-scatter plots for biophysical profiles of MCF 7 cells: Electrical Deformability Index (left), Membrane Opacity (middle), Nucleus Opacity (right) according to various embodiments.

FIG. 26 shows (A) graph plots of a neutrophil purity comparison between DFF and Percoll; aggregates formation comparison between DFF and Percoll; and PBMC isolation efficiency; (B) 2D-scatter plots of neutrophil nucleus opacity versus cell size; (C) biophysical properties (cell size, deformability, membrane opacity, and nucleus opacity) graph plots based on impedance measurements; and (D) a 3D scatter plot for DFF-isolated neutrophils from healthy and T2DM subjects.

FIG. 27 shows (A) 2D-scatter plots for electro-mechano properties for Neutrophils, Monocytes and Lymphocytes; and (B) UMAP analysis for Healthy and T2DM Neutrophils, Monocytes, and lymphocytes.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention. Embodiments described below in context of the device/apparatus/system are analogously valid for the respective methods, and vice versa. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail.

“About”, as used herein in connection with numerical values refers to the referenced numerical value ±10% or ±5%.

Conventional neutrophil studies require surface antigen labelling to assess inflammation status which are laborious and expensive. Various microfluidic approaches have been proposed to study neutrophil functions (e.g. neutrophil chemotaxis or phagocytosis), and cellular biophysical properties linked to cell biology and pathology, whereby cell deformability has been used as a biophysical marker for identifying cell diseases and cellular states⁶, and has been used for lymphocyte activation studies⁷ and sepsis diagnosis⁸. One technique for measuring cell deformability is atomic force microscope (AFM) which characterizes cell mechanics by using a mechanical probe to induce cell deformation, however, this suffers from low throughput (few cells/hr). Another conventional technique is impedance cytometry utilizing constricted detection channel (smaller than cell size) to deform cells and measuring cell deformability and impedance based on transit times of single cells traversing through the channel, however, these techniques suffers from low throughput and the constricted channels are prone to clogging issues. Additionally, the electrical measurement during cell squeezing through the constriction channel may not reflect non-squeezed/native state properties. While non-constriction based deformability measurement methods exist, they rely on and necessitate the use of expensive high-speed camera (>10000 fps) and sophisticated optical setups to quantify morphological changes in particle/cell shape.

Accordingly, there is provided herein a method and system employing a specifically configured microfluidic device or platform to electrically measure single-cell multi-parametric biophysical properties in a label-free and optics-free manner that alleviates the problems of conventional cell profiling techniques. The method, system, and device disclosed herein may be configured for point-of-care testing applications such as multi-parametric profiling of cells (e.g. neutrophils) based on cell deformability and dielectric properties of cell membranes. In particular, the method, system, and device disclosed herein may be used for high throughput (>1000 cells/min), label-free, and optics-free biophysical profiling (e.g. cell size, cell deformability, membrane impedance) of single cells.

This development was made possible by at first characterizing the cells (for example, HL60 (Leukemia cell line)) and electrically mapping out their biophysical parameters with different stimuli. In particular, a numerical impedance simulation was utilized to characterize the impedance signal for particles of different shapes (aspect ratios) and observe strong correlations with conventional image-based deformability index (major/minor axis). An “electrical deformability index” based on differential impedance signatures of native and deformed cells (a ratio between impedance response measured at native state to impedance response measured at deformed state, preferably at frequency range of 0.1-1 MHz) was defined to study the cells (e.g. HL60) of different stiffness.

As a proof-of-concept for clinical utilization, primary human neutrophils treated with different biochemical stimuli were profiled to demonstrate multi-functional cell profiling based on biophysical properties. To alleviate clogging problems typically found in constriction-based deformability cytometers, as well as alleviating other aforementioned problems of conventional techniques, it was advantageously found that the method, system, and microfluidic device disclosed herein were able to 1) achieve single stream particle focusing, 2) deform cells using a sheath stream at a junction, and/or 3) electrically characterize the changes in one or more biophysical properties (e.g. cell size, deformability and membrane impedance), using an electrode arrangement/configuration at a junction (e.g. convergence of intersecting flow channels).

Collectively, the results reported herein demonstrate the potential of the developed method, system and microfluidic device disclosed herein to offer multi-parametric single cell mechano-phenotyping without any complicated optical setups. This represents a key step towards identifying novel cellular biomarkers and the development of next generation label-free cytometers for rapid clinical immunoprofiling that may elucidate the relevance between single-cell biophysical characteristics and neutrophil functions in various diseases such as cancer, urinary tract infection (UTI) as well as sepsis. It is also envisioned that the various embodiments may be further used for quantitative detection of cell (or neutrophil) responses as point-of-care testing for infectious diseases (e.g. tuberculosis and COVID-19). The cell analysis and continuous collection of eluted cells may greatly facilitate downstream bioanalysis to elucidate novel associations between biophysical cellular properties and antigen-specific responses.

As will be appreciated, it may also be possible to integrate the developed method, system and microfluidic device disclosed herein with other flow-based single-cell measurement modalities for automated clinical diagnostics.

The methods of the various embodiments allow optics-free and label-free quantitative cell profiling based on impedance-deformability cytometry using a developed microfluidic device and may include the following steps:
forming a single stream of cells in a sample flow channel of the microfluidic device by perfusing a fluid sample containing the cells through a particle focusing region of the microfluidic device, wherein the particle focusing region is upstream of a detection region of the microfluidic device, wherein the sample flow channel extends through the particle focusing region and the detection region;
measuring an impedance response of each cell by a first pair of electrodes disposed along the sample flow channel within the detection region;
deforming each cell within a cell deformation zone along the sample flow channel within the detection region, wherein the cell deformation zone is downstream of the first pair of electrodes;
measuring an impedance response of each cell after deformation by a second pair of electrodes disposed along the sample flow channel within the detection region of the microfluidic device, wherein the second pair of electrodes is downstream of the cell deformation zone;
determining one or more quantitative relationships among impedance responses from one or a combination of the first and second pair of electrodes to quantify one or more biophysical properties of each cell for profiling of said cell in said fluid sample.

The term “optics-free”, as used herein, is meant to include methods, systems and microfluidic devices that may be utilised for cell profiling without the use of optic setups that may include a camera or laser, i.e., “optics-free” microfluidics. Accordingly, the methods, systems and microfluidic devices according to the various embodiments may be free of optic setups for sensing or detecting or measuring the cells, and may quantify the one or more biophysical properties of the cells without using visual inputs or data or information (e.g. images or videos or screen capture etc).

The cells profiled may be blood cells, such as white blood cells, namely myelocytes and/or lymphocytes, with neutrophils being used as a particular example to demonstrate the feasibility of the method herein. The methods described and exemplified are thus mainly methods that allow activated neutrophil profiling. However, other cell types in the size range of 8 to 40 μm may also be profiled, including but not limited to cancer cells, stem cells and other mammalian cells.

The term “blood cells”, as used herein, is meant to include platelets, red blood cells and leukocytes (white blood cells). Types of leukocytes can be classified in standard ways. Two pairs of broadest categories classify them either by structure (granulocytes or agranulocytes) or by cell lineage (myeloid cells or lymphoid cells). These broadest categories can be further divided into the five main types: neutrophils, eosinophils, basophils, monocytes, and lymphocytes. These types are distinguished by their physical and functional characteristics. Monocytes and neutrophils are phagocytic. Further subtype classification is well-known in the art.

The term “neutrophils” used herein, has its general meaning in the art and are a category of white blood cells characterized by the presence of granules in their cytoplasm. They may also be called polymorphonuclear leukocytes (PMN, PML, or PMNL) because of the varying shapes of the nucleus, which is usually lobed into three segments. This distinguishes them from the mononuclear agranulocytes.

In various embodiments, the method does not include a labelling step, wherein at least one type of blood cells is labelled with a marker molecule. Accordingly, the method of the various embodiments may be free of a labelling step and the cells may be free of any labels. The term “labelling”, as used herein, denotes the attachment or incorporation of one or more detectable markers (or “labels”) into a cell used in the various embodiments. The terms “marker molecule” or “detectable marker”, as interchangeably used herein, refer to any compound that includes one or more appropriate chemical substances or enzymes, which directly or indirectly generate a detectable compound or signal in a chemical, physical or enzymatic reaction. As used herein, the term is to be understood to include both the labels as such (i.e., the compound or moiety bound to the protein and/or peptide) as well as the labelling reagent (i.e., the compound or moiety prior to the binding with the peptide or protein). All these types of labels are well established in the art. Thus, the method disclosed herein may be termed a “label-free” method.

In various embodiments, the sample used may be a blood sample. This sample may be processed before it is used in the method, e.g. by subjecting to various separation, purification and conservation treatments, all of which are well-known to those skilled in the art. For example, the red blood cells may be lysed or otherwise separated from the remainder of the blood sample to increase the concentration of white blood cells and reduce the red blood cell background.

In various embodiments, additional separation steps may be carried out to separate subpopulations of white blood cells. For example, for white blood cells which occur in low total numbers in comparison to the predominant neutrophil populations, such as basophils, eosinophils and monocytes, it may be beneficial to separate not only the red blood cells and platelets, but also the neutrophils to allow profiling of these cells. The same applies when profiling neutrophils. In such embodiments, it may not be essential but still beneficial to separate the subpopulation not to be assessed from the one being profiled prior to carrying out the methods described herein. Means for separating different subpopulations from each other are well known in the art.

The term “Sample” or “fluid sample”, as used herein, generally includes biological samples such as tissues and bodily fluids. “Bodily fluids” may include, but are not limited to, blood, serum, plasma, saliva, cerebral spinal fluid, pleural fluid, tears, lactal duct fluid, lymph, sputum, urine, amniotic fluid, and semen. A sample may include a bodily fluid that is “acellular”. An “acellular bodily fluid” includes less than about 1% (w/w) whole cellular material. Plasma or serum are examples of acellular bodily fluids. A sample may include a specimen of natural or synthetic origin. “Plasma”, as used herein, refers to acellular fluid found in blood. “Plasma” may be obtained from blood by removing whole cellular material from blood by methods known in the art (e.g., centrifugation, filtration, and the like). As used herein, “peripheral blood plasma” refers to plasma obtained from peripheral blood samples. “Serum”, as used herein, includes the fraction of plasma obtained after plasma or blood is permitted to clot and the clotted fraction is removed. Thus, a serum sample is also acellular. In contrast, the term “whole blood”, as used herein, means blood per se collected from a subject such as a human and containing unseparated blood cells. Further, a sample obtained by diluting the whole blood with an appropriate buffer or the like, and/or by adding an additive, such as an anticoagulant or a protease inhibitor, may be used as the whole blood sample. A whole blood sample includes blood cells, such as platelets, red blood cells and leukocytes. Since the methods disclosed herein are related to profiling of cells, in particular lymphocytes, sample types that typically contain cells are preferred in the sense of the various embodiments.

In various embodiments, the method and system according to various embodiments herein may be used to assist with profiling subjects with type-2 diabetes mellitus. Accordingly, the fluid sample may be obtained from a subject with type-2 diabetes mellitus.

In various embodiments, the method according to various embodiments herein may be used to assist with profiling subjects recovered from COVID-19. Accordingly, the fluid sample may be obtained from a subject recovered from COVID-19 or has previously been infected with and recovered from COVID-19.

The sample may be a finger prick sample or a sample generated from venipuncture. “Finger prick sample”, as used herein, is a sample of blood consisting of one drop collected from a finger (tip). Preferably, the sample is collected by a small fingertip lancet device capable of painlessly piercing the skin to cause fresh blood flow. Once a hanging droplet of blood is available on the finger a collector may be applied to the droplet and blood may be absorbed by the collector. The volume of the finger prick sample may range from 10-500 μL, preferably 50-300 μL and more preferably 80-150 μL. Alternatively, the blood drop may also be collected from other parts of the body, such as the ear lobe. The features described for finger prick samples similar apply to such blood drop samples collected from other regions of the body.

In various embodiments, the cells may be neutrophils.

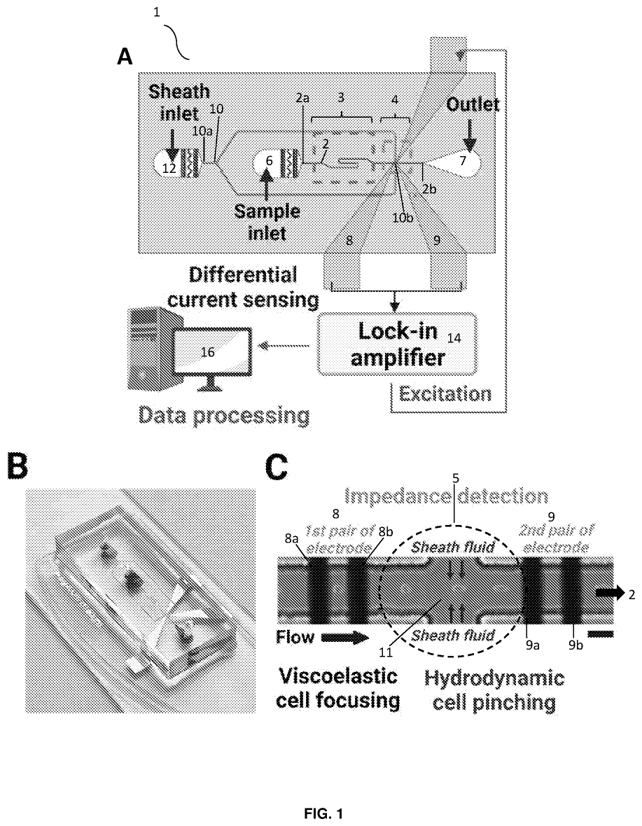

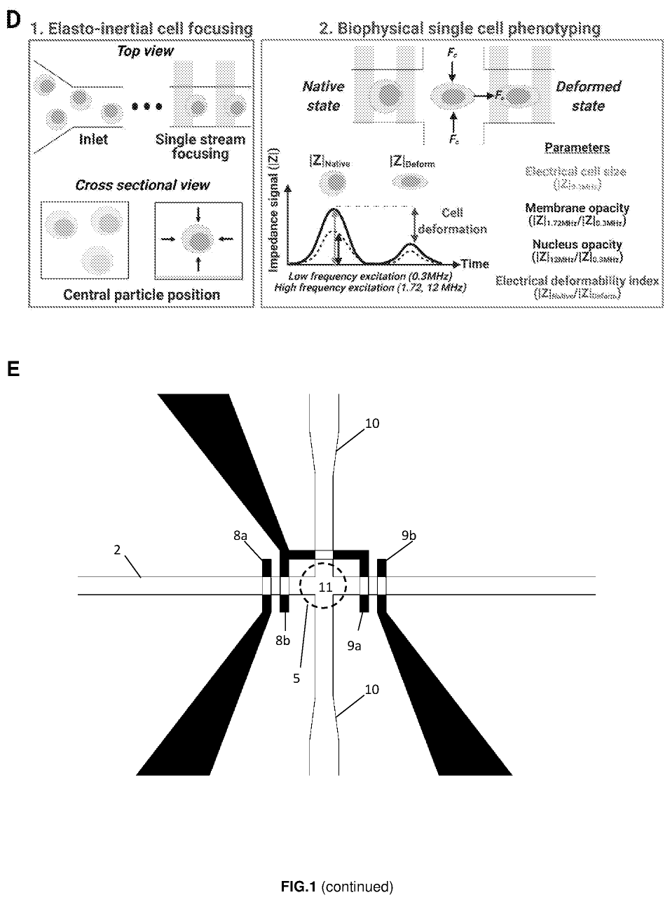

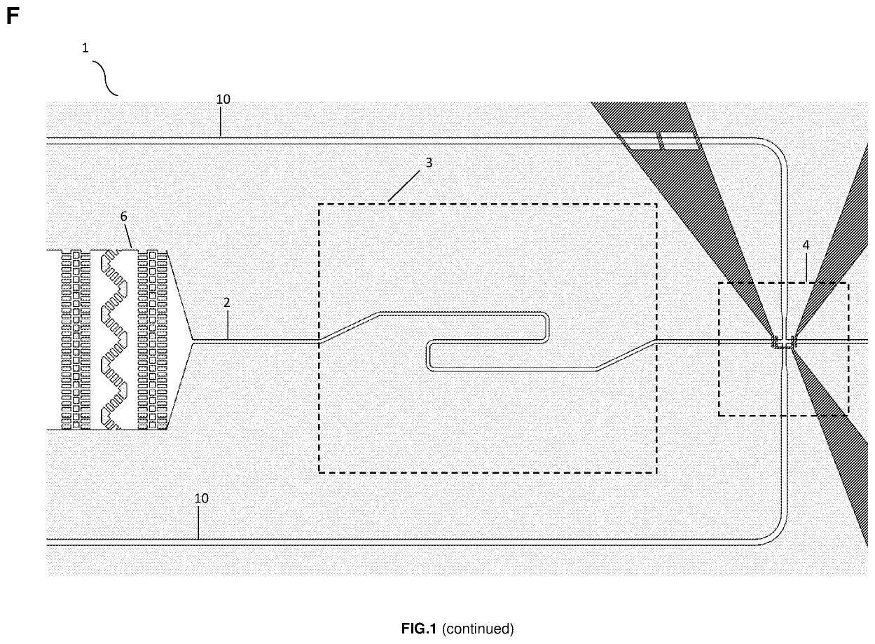

In various embodiments, the method uses a microfluidic device (1) that may include a sample flow channel (2), a particle focusing region (3), and a detection region (4) with a cell deformation zone (5), as shown in FIGS. 1A, 1B, 1C, 1E and 1F.

As used herein, the expression “microfluidic device” means a physical element that enables the control and manipulation of fluids that are geometrically constrained to a small, typically sub-millimeter scale. “Microfluidic device”, as used herein, refers to a device that includes one or more microfluidic flow channels designed to carry, store, mix, react, and/or analyze liquid samples, typically in volumes of less than one milliliter. Representative examples of materials that may be used to make microfluidic devices include, but are not limited to, silicone rubber, glass, plastic, silicon and metals, preferably the microfluidic device may be made of polydimethylsiloxane (PDMS).

Dimensions of microfluidic devices and the flow channels thereof, and the materials for constructing microfluidic devices and methods for constructing microfluidic devices are readily known to those skilled in the technical field^{30, 32-37}. As used herein, the term “flow channel” means a tubular passage for liquids.

In various embodiments, the sample flow channel may have at least a first end (2a) and a second end (2b), wherein said sample flow channel has the one inlet port (6) at or near said first end and an outlet port (7) at or near said second end. The sample flow channel may include one sample inlet and one sample outlet. In some embodiments, the sample inlet may be located proximate to the centre of the device, and the sample outlet may be located proximate to an outer wall of the device, as shown in FIG. 1A, B.

The term “inlet port”, as used herein, is defined as the end of the flow channel, which may be first contacted with the fluid to be introduced or perfused therein. On the contrary, the term “outlet port” refers to the end of the flow channel, which releases the fluidic material from the microfluidic device, for example into a container it may be connected to store the recovered fluid, which may allow further purification or analysis of the recovered fluid.

In various embodiments, the sample flow channel (2) may have a diameter that is larger than the diameter of the cells. In one embodiment, the sample flow channel (2) may be a square channel with a width of 30 to 50 μm, preferably about 45 μm, and a height of 30 to 50 μm, preferably about 45 μm. The sample channel may have a total length of 1 to 2 cm, preferably about 1.4 cm.

In the above embodiments, the methods using the microfluidic devices may typically include introducing or perfusing the sample into the sample inlet port at a sample flow rate. In one embodiment, the sample flow rate may be in the range of 1 to 20 μL/min.

In various embodiments, the particle focusing region (3) may include a convoluting or winding path section of the sample flow channel (2) such that the channel includes two or more turns to extend the length of the channel within the particle focusing region (3). This convolution or winding of the sample flow channel may assist in forming a single stream of cells that are aligned in the center of the sample flow channel (2).

In various embodiments, viscoelastic focusing may be employed for the particle focusing, whereby the sample flow channel in the particle focusing region may be in the form of a square channel of 30 to 50 μm, preferably 45 μm, in width and height, and 1-2 cm, preferably 1.2 cm, in length for single cell alignment.

In various embodiments, the fluid sample may include a viscoelastic medium, preferably 0.5-3% poly(ethylene oxide) or other polymers, in phosphate-buffered saline.

Accordingly, in the method of the various embodiments, forming the single stream of cells in the sample flow channel may include aligning the cells into the single stream of cells along a center of the sample flow channel by viscoelastic focusing effect. The aligning of the cells into the single stream of cells along the center of the sample flow channel by viscoelastic focusing effect may include perfusing the fluid sample containing the cells through a winding path section of the sample flow channel forming the particle focusing region of the microfluidic device, wherein the winding path section of the sample flow channel has a pre-determined length based on a viscoelasticity of the viscoelastic medium in order to complete alignment of the cells into the single stream of cells by viscoelastic focusing effect within the winding path section.

In various embodiments, the detection region may include a first pair of electrodes (8) disposed along the sample flow channel, the cell deformation zone (5), and a second pair of electrodes (9) disposed along the sample flow channel. In one embodiment, the detection region may be downstream of the particle focusing region. In one embodiment, the cell deformation zone may be downstream of the first pair of electrodes and upstream of the second pair of electrodes (i.e, the second pair of electrodes is downstream of the cell deformation zone). That is, the first and second pair of electrodes may be respectively arranged before and after the cell deformation zone.

In various embodiments, for each pair of the electrodes, one electrode (8b, 9a) may operate to apply an excitation signal at one or more frequencies, and the other electrode (8a, 9b) may measure an impedance response, as shown in FIG. 1E. In one embodiment, the pair of electrodes may be coplanar electrodes. In one embodiment, the first pair of electrodes may be substantially parallel to each other, and the second pair of electrodes may be substantially parallel to each other. The coplanar electrodes may be arranged in a side-by-side manner along a same side (e.g. underneath) the sample flow channel and in direct contact with fluid sample.

In various embodiments, the width of each electrode may be about 20-30 μm (i.e. slightly larger than the size of the cell to be profiled) with about a 10-20 μm gap or spacing between the electrodes in each pair of electrodes. The spacing between the first pair of electrodes and the second pair of electrodes (i.e, the two detection sites for native and deformed cell states) on either side of the cell deformation zone may be about 150-250 μm. In this regard, if a smaller spacing (<150 μm) between the first pair of electrodes and the second pair of electrodes is employed, the pinching at the junction in the cell deformation zone may adversely affect the electrical detection, alternatively a larger spacing (>250 μm) is also not favourable as deformed cells may relax back to native states.

Accordingly, in the method disclosed herein, the impedance response of each cell prior to passing through the cell deformation zone may be measured by the first pair of electrodes disposed along the sample flow channel within the detection region. In a similar manner, in the method disclosed herein, the impedance response of each cell after passing through the cell deformation zone may be measured by the second pair of electrodes disposed along the sample flow channel within the detection region. In the former, each cell is in its native state before deformation, whereas in the latter each cell has been hydrodynamically stretched either by compressive or shear forces into its deformed state.

In various embodiments, in the method disclosed herein, the impedance response measured by each pair of the first and second pair of electrodes may include measuring a multi-frequency impedance response, wherein a multi-frequency excitation signal may be applied by each pair of the first and second pair of electrodes for measuring the multi-frequency impedance response. The frequencies may be in a range of about 0.1 MHz to 20 MHz.

In various embodiments, the multi-frequency impedance response may include impedance signals at two or more different frequencies. In one embodiment, the two or more frequencies include a first impedance signal at a first frequency in a range of about 0.1 MHz to 0.5 MHz, and a second impedance signal at a second frequency in a range of about 1 MHz to 5 MHz.

In another embodiment, the two or more frequencies may include the first impedance signal at a first frequency of about 0.3 MHz and the second impedance signal at a second frequency of about 1.7 MHz.

In another embodiment, the multi-frequency impedance response may include impedance signals at three or more different frequencies. In one embodiment, the three or more frequencies include a first impedance signal at a first frequency in a range of about 0.1 MHz to 0.5 MHz, a second impedance signal at a second frequency in a range of about 1 MHz to 5 MHz, and a third impedance signal at a third frequency in a range of about 5 MHz to 20 MHz.

In one embodiment, the three or more frequencies may include the first impedance signal at a first frequency of about 0.3 MHz, the second impedance signal at a second frequency of about 1.7 MHz, and the third impedance signal at a third frequency of about 12 MHz.

After the impedance response is measured via the first pair of electrodes, the cell flows through the cell deformation zone and are deformed according to the method disclosed herein. In one embodiment, the deformation of the cell may be achieved without the cell contacting with the walls of the sample flow channel, that is without a constriction channel or a narrowing of the flow channel to contact with and deform the cells through squeezing or constriction. Accordingly, the sample flow channel passing through the cell deformation zone may have a diameter that is larger than the diameter of the cells.

In various embodiments of the method disclosed herein, deformation of the cell may include hydrodynamically deforming each cell within the cell deformation zone by perfusing a sheath fluid along a sheath flow channel (10) that converges in an intersecting manner with the sample flow channel at the cell deformation zone. This convergence and point of intersection may be termed as a junction (11).

The term “hydrodynamically deforming” used herein, refers to the use of hydrodynamic forces or stresses from sheath fluid or intrinsic fluid viscoelastic properties, for inducing mechanical deformation of the cells.

In various embodiments, the sheath flow channel may have at least a first end (10a) and a second end (10b), wherein said sheath flow channel has the one inlet port (12) at or near said first end and where the second end may be the point at which the sheath flow channel converges in an intersecting manner with the sample flow channel. Accordingly, the microfluidic device disclosed herein may represent a 2-inlet 1-outlet device.

In one embodiment, the sheath flow channel converges in an intersecting manner with the sample flow channel to form a junction at the cell deformation zone.

In various embodiments, the sheath inlet may be located proximate to an outer wall of the device and in horizontal alignment with the sample inlet, and the second end of the sheath flow channel being located within the detection region and specifically within the cell deformation zone, as shown in FIGS. 1A, C and E. In one embodiment, the sheath inlet, sample inlet and outlet may be in horizontal alignment, with the sheath inlet and outlet being located proximate to opposing outer walls and the sample inlet being located therein between and proximate to the centre of the device.

The term “sheath fluid”, as used herein, refers to a variety of fluids, including aqueous or nonaqueous fluids and/or fluids that may include additional material components, e.g., soluble chemical components or suspensions or emulsions of at least partially insoluble components. Preferably, the sheath fluid may be a buffer, preferably a compatible with blood cells, such as phosphate-buffered saline (abbreviated PBS) and other well-known buffers. In one embodiment, the sheath fluid consists only of a viscoelastic medium, preferably poly(ethylene oxide).

In various embodiments, the sheath flow channel may be furcated into two or more branches of equal length. In one embodiment, the sheath flow channel may be bifurcated into two branches or split flow paths of equal length that converge in an intersecting manner with the sample flow channel at the cell deformation zone from two opposite sides of the sample flow channel. The convergence and intersecting of the two split flow paths with the sample flow channel apply a hydrodynamic force for deforming each cell flowing through the cell deformation zone along the sample flow channel.

In one embodiment, the two split flow paths converge in an intersecting manner with the sample flow channel to form a cross-junction.

As used herein, the term “furcated” means divided into branches. As used herein, the term “branch” means a limited part of a larger or more complex body, i.e., a smaller flow channel emerging from a primary flow channel or entering or re-entering a primary flow channel.

At the junction or cross-junction where the sample and sheath streams converge and intersect, the sheath flow channel may have the same dimensions as the sample flow channel for simplicity. Accordingly, in various embodiments, the sheath flow channel may have a width of 50 to 100 μm, preferably about 66 μm, and a height of 30 to 50 μm, preferably about 45 μm, and/or has a total length of 1.5 to 2 cm, preferably about 1.7 cm. Prior to the junction or cross-junction the width of the sheath flow channel may narrow to match the width of the sample flow channel, as shown in FIG. 1E.

In various embodiments, the method may additionally include introducing or perfusing the sheath fluid into the sheath inlet at a sheath flow rate. In one embodiment, the sheath flow rate may be in the range of 1 to 40 μL/min.

In this regard, both the sample and sheath fluid flow rate may be parameters that impact on the deformation of the cells in the deformation zone. Accordingly, in various embodiments of the method, the fluid sample may be perfused at a sample flow rate and the sheath fluid may be perfused at a sheath flow rate, wherein the sheath flow rate may be higher than the sample flow rate. In one embodiment, the sample flow rate may be in the range of 1 to 20 μL/min, and the sheath flow rate may be in the range of 1 to 40 μL/min, preferably the sheath to sample flow rate ratio may be 3:1 to 5:1.

In various embodiments of the method, the fluid sample may include a viscoelastic medium, preferably poly(ethylene oxide), in phosphate-buffered saline, and the sheath fluid consists of a viscoelastic medium, preferably poly(ethylene oxide) in phosphate-buffered saline.

The sample and sheath fluid may be introduced to their respective flow channels by means of a syringe or other means. Other means for introducing a sample into a flow channel of a microfluidic device include microdispensers, e.g., droplet dispensers, such as, for example, injection nozzles; in-channel dispensers, e.g., metering dispensers. Means for introducing samples into a flow channel of a microfluidic device are discussed in greater detail by Nguyen et al. and Li et al^{30, 31}. Nguyen et al. discusses microdispensers in detail. Li et al. discusses microdispensers in detail.

The sample and sheath fluid may be driven by the force of capillary action. Alternatively, the sample may be driven by a pump, by electrical forces, or by other means for driving samples.

The same applies to the sheath fluid, which is however, typically not driven by capillary action but rather by active means, such as a pump. Suitable pumps include, but are not limited to, rotary (centrifugal) pumps; peristaltic pumps; and ultrasonic pumps. Electrical forces include, but are not limited to, electrohydrodynamic forces; electrokinetic forces, e.g., electrophoresis, electro-osmosis; and surface tension driven, e.g., electrowetting, electrowetting on dielectric surface. Means for driving samples are discussed in greater detail by Nguyen et al.³⁰, Erickson et al.³⁸, Grover et al.³⁹, Hunt et al.⁴⁰ and Bersano-Begey et al.⁴¹. Nguyen et al.³⁰ discusses microvalves, micropumps, microflow sensors, microfilters and microseparators in detail.

In various embodiments, the Reynolds number (Re) of the sample flowing through the flow channel may be about 0.001-10, preferably, 0.01 (at 2 μL/min using 2.5% PEO). As used herein, the expression “Reynolds number” means pUDH/p; wherein p represents density of a liquid; U represents velocity of the liquid; DH represents hydraulic diameter given by 2WH/(W+H), W represents width, H represents height, and p represents viscosity of the liquid.

In the method of the various embodiments, multi-frequency impedance responses may be measured by one or a combination of the first pair of electrodes and the second pair of electrodes to respectively capture different single-cell properties before and after cell deformation at the deformation zone. Accordingly, the methods disclosed herein may include quantitatively profiling the cells, preferably activated neutrophils, by determining one or more quantitative relationships among impedance responses measured from one or a combination of the first and second pair of electrodes to quantify one or more biophysical properties of each cell for profiling of said cell in said fluid sample. In various embodiments, the one or more biophysical properties are quantified simultaneously.

In various embodiments, the one or more biophysical properties may be selected from membrane opacity; nucleus opacity; cell size; and deformability index. In another embodiment, four or more biophysical properties are quantified and include membrane opacity; nucleus opacity; cell size; and deformability index. The term “biophysical properties”, can be used interchangeably herein with “biophysical characteristics” and “biophysical features”.

Electrical opacity may be calculated as the ratio of impedance signal magnitude at high frequency (>1 to 20 Mhz) to impedance signal magnitude at low frequency (0.1 to 0.5 MHz). In various embodiments, the measurement at the low-frequency range may be combined with any one of the two higher frequency ranges in order to allow the determination of membrane properties or nucleus properties.

In various embodiments, electrical opacity may reflect cell membrane electrical properties. Accordingly, in various embodiments, determining one or more quantitative relationships among impedance responses from the first and/or second pair of electrodes may include determining a ratio of the second impedance signal at the second frequency and the first impedance signal at the first frequency to quantify a membrane opacity of each cell as one of the one or more biophysical properties for said cell.

In various embodiments, electrical opacity may reflect electrical properties of the cell nucleus. Accordingly, in various embodiments, determining one or more quantitative relationships among impedance responses from the first and/or second pair of electrodes may include determining a ratio of the third impedance signal at the third frequency and the first impedance signal at the first frequency to quantify a nucleus opacity of each cell as one of the one or more biophysical properties for said cell.

To measure cell size, the impedance response at the first frequency correlates to the physical size of cells. Further, by measuring the impedance responses before and after deformation, the electrical deformability index either may be measured by differencing or normalizing impedance signals between the native and deformed states.

In various embodiments, the impedance measurement may be carried out at a low frequency in the range of 0.1 to 0.5 MHz, to determine cell size. Accordingly, in various embodiments, determining the first impedance signal at the first frequency of the first pair of electrodes may quantify the cell size of each cell.

In various embodiments, determining one or more quantitative relationships among impedance responses from one or a combination of the first and second pair of electrodes may include determining a ratio of the impedance response of the first pair of electrodes and the impedance response of the second pair of electrodes to quantify a deformability index of each cell as one of the one or more biophysical properties for said cell. In particular, determining one or more quantitative relationships among impedance responses from one or a combination of the first and second pair of electrodes may include determining a ratio of the first impedance signal (native state) at the first frequency from the first pair of electrodes to the first impedance signal (deformed state) at the first frequency from the second pair of electrodes to quantify a deformability index of each cell as one of the one or more biophysical properties for said cell.

In addition, the higher frequency range indicated, namely >10 to 20 MHz may also be used to determine properties of intracellular structures, for example to assess phagocytosis. It has also been found that this high-frequency range allows profiling neutrophils to identify those undergoing NETosis, as this is accompanied by nuclear structural changes such as nuclear swelling, nuclear breakdown.

After profiling of the cells, the cells may be discarded or collected in containers connected to the respective outlet port. This allows further use and/or analysis of the cells.

Various embodiments seek to provide an optics-free and label-free system for single-cell profiling using a developed microfluidic device disclosed herein and a computing system, wherein the system may include: a microfluidic device including a sample flow channel extending through a particle focusing region and a detection region, wherein the detection region may include a cell deformation zone, a first pair of electrodes and a second pair of electrodes, wherein the first and second pair of electrodes are respectively arranged before and after the cell deformation zone; and a computing system including: a memory; and at least one processor communicatively coupled to the memory and the first and second pair of electrodes of the microfluidic device, wherein the computing system may be configured to:
(i) measure an impedance response of each cell flowing past the first pair of electrodes disposed along the sample flow channel within the detection region;
(ii) measure an impedance response of each cell after deformation flowing past the second pair of electrodes disposed along the sample flow channel within the detection region; and
(iii) determine one or more quantitative relationships among impedance responses from one or a combination of the first and second pair of electrodes to quantify one or more biophysical properties of each cell for profiling of said cell.

All embodiments described above in the context of the method and concerning the microfluidic device are similarly applicable to the system and the operations that the computing system may be configured to carry out.

In various embodiments, the computing system corresponds to the method for single-cell profiling as disclosed herein, therefore, various functions or operations configured to be performed by the least one processor may correspond to various steps of the method as disclosed herein according to various embodiments, and thus need not be repeated in detail with respect to the system for clarity and conciseness. In other words, various embodiments described herein in the context of the methods are analogously valid for the respective systems, and vice versa.

The computing system, for performing the operations/functions of the methods described herein may be specially constructed for the required purposes or may include a general-purpose computer or other devices selectively activated or reconfigured by a computer program stored in the computer.

It will be appreciated by a person skilled in the art that the at least one processor may be configured to perform the required functions or operations through set(s) of instructions (e.g., software modules) executable by the at least one processor to perform the method that may be automated by the computing system.

The term “computing system” encompasses all kinds of apparatus, devices, and machines for processing data, including by way of example a programmable processor, a computer, a system on a chip, or multiple ones, or combinations, of the foregoing. The “computing system” may include special purpose logic circuitry, e.g., an FPGA (field programmable gate array) or an ASIC (application specific integrated circuit). The “computing system” may also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, or code) may be written in any form of programming language, including compiled or interpreted languages, declarative or procedural languages, and it may be deployed in any form, including as a stand-alone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program, or in multiple coordinated files (e.g., files that store one or more modules, sub programs, or portions of code). A computer program may be deployed to be executed on one computer or on multiple computers that are located at one site or distributed across multiple sites and interconnected by a communication network.

Some of the processes and logic flows described in this specification may be performed by one or more programmable processors executing one or more computer programs to perform actions by operating on input data and generating output. The processes and logic flows may also be performed by, and apparatus may also be implemented as, special purpose logic circuitry, e.g., an FPGA (field programmable gate array) or an ASIC (application specific integrated circuit).

In various embodiments of the system, the system may further include a lock-in amplifier, impedance analyzer or data acquisition system communicatively coupled between the computing system and the first and second pair of electrodes of the microfluidic device, wherein each pair of the first and second pair of electrodes may be configured to apply a multi-frequency excitation signal to each cell, wherein the computing system may be configured to measure, via the lock-in amplifier, a multi-frequency impedance response by each pair of the first and second pair of electrodes. In various embodiments, the system may include one or more transimpedance amplifiers, whereby one electrode (8a,9b) of each pair may be connected to a transimpedance amplifier to convert impedance responses for the lock-in amplifier.

In various embodiments of the system, the computing system may be configured to: measure, via the lock-in amplifier, the multi-frequency impedance response including impedance signals at two or more frequencies, wherein the two or more frequencies are in a range of about 0.1 MHz to 20 MHz. In one embodiment, the two or more frequencies may include a first impedance signal at a first frequency in a range of about 0.1 MHz to 0.5 MHz, and a second impedance signal at a second frequency in a range of about 1 MHz to 5 MHz. In another embodiment, the two or more frequencies may include the first impedance signal at a first frequency of about 0.3 MHz and the second impedance signal at a second frequency of about 1.7 MHz.

In various embodiments of the system, the computing system may be configured to: measure, via the lock-in amplifier, the multi-frequency impedance response including impedance signals at three or more frequencies, wherein the three or more frequencies are in a range of about 0.1 MHz to 20 MHz. In one embodiment, the three or more frequencies may include a first impedance signal at a first frequency in a range of about 0.1 MHz to 0.5 MHz, a second impedance signal at a second frequency in a range of about 1 MHz to 5 MHz, and a third impedance signal at a third frequency in a range of about 5 MHz to 20 MHz. In another embodiment, the three or more frequencies may include the first impedance signal at a first frequency of about 0.3 MHz, the second impedance signal at a second frequency of about 1.7 MHz, and the third impedance signal at a third frequency of about 12 MHz.

In various embodiments of the system, the computing device may be configured to determine a ratio of the second impedance signal at the second frequency and the first impedance signal at the first frequency to quantify a membrane opacity of each cell as one of the one or more biophysical properties for said cell.

In various embodiments of the system, the computing device may be configured to determine a ratio of the third impedance signal at the third frequency and the first impedance signal at the first frequency to quantify a nucleus opacity of each cell as one of the one or more biophysical properties for said cell.

In various embodiments of the system, the computing device may be configured to quantify a cell size of each cell based on the first impedance signal at the first frequency of the first pair of electrodes.

In various embodiments of the system, the computing system may be configured to determine a ratio of the impedance response of the first pair of electrodes and the impedance response of the second pair of electrodes to quantify a deformability index of each cell as one of the one or more biophysical properties for said cell.

Various embodiments also encompass the microfluidic devices disclosed herein. In one embodiment, there is provided a microfluidic device for single-cell profiling, including: a first flow channel to form a fluid pathway for allowing a fluid sample including cells to flow from a sample inlet to an outlet, wherein the first flow channel may include a particle focusing region;
a second flow channel to form a fluid pathway for allowing a sheath fluid to flow from a sheath fluid inlet to a junction in a cell deformation zone, wherein the second flow channel intersects with the first flow channel to form the junction in a cell deformation zone;
two pairs of electrodes arranged adjacent to the junction, wherein the at least two pairs of electrodes and the junction define a detection region,
wherein the two pairs of electrodes may include a first pair of electrodes and a second pair of electrodes, and
wherein the first pair of electrodes extend across the first flow channel upstream of the junction, and wherein the second pair of electrodes extend across the first flow channel downstream of the junction.

In this regard, the “first flow channel” may be used interchangeably with a “sample flow channel”, and the “second flow channel” may be used interchangeably with a “sheath flow channel”.

In various embodiments of the microfluidic device, the second flow channel may be bifurcated into two split-flow pathways that converge in an intersecting manner with the first flow channel to form the junction, wherein the junction is a cross-junction.

In various embodiments of the microfluidic device, the particle focusing region may be upstream of the detection region, wherein the first flow channel extends through the particle focusing region, the cell deformation zone and the detection region.

In various embodiments of the microfluidic device, the detection region may include the cell deformation zone along the first flow channel, wherein the cell deformation zone is downstream of the first pair of electrodes.

In various embodiments of the microfluidic device, the second pair of electrodes may be downstream of the cell deformation zone.

In various embodiments of the microfluidic device, the particle focusing region may be positioned upstream of the detection region.

In various embodiments of the microfluidic device, the first pair of electrodes may be substantially parallel to each other, and the second pair of electrodes may be substantially parallel to each other.

Such microfluidic devices may be used for profiling of neutrophils, preferably in setups in which the respective other cell population is removed before the sample is introduced in the sample inlet of the device.

According to another aspect, the optics-free and label-free system for single-cell profiling may be in the form of an integrated impedance cytometer including the microfluidic device disclosed herein and further including one or more, preferably all of, (1) a microchip, (2) an impedance analyzer, and (3) fluidic pumps connected to the sample and sheath fluid inlet to enable perfusion control, wherein the microchip is interfaced with the impedance analyzer and the fluidic pumps and wherein the impedance analyzer is connected to the electrodes to enable impedance measurements. The microchip and the impedance analyzer may together serve as the computing system to operate the cytometer and carry out one or more of the method steps disclosed herein.

In various embodiments, the realization of an integrated platform featuring continuous, optics-free and label-free cell impedance profiling on a single chip may have a wide range of clinical applications including immunology studies, cancer diagnostic, and point-of-care and immune health profiling. This technology may offer single-step optics-free and label-free cell impedance analysis with minimal human handling. It may also be possible to integrate the developed platform with other cell-based platforms for further multi-parametric studies. With all the features of this integrated platform, it may be developed into a product for immunology research and point-of-care testing market.

All embodiments disclosed above in relation to the methods similarly apply to the microfluidic device and the system (e.g. an impedance cytometer) and vice versa.

Various embodiments are further illustrated by the following examples. However, it should be understood, that various embodiments not limited to the exemplified embodiments.

In the following, device configuration, fabrication and operation of the exemplified embodiments are described.

An exemplified embodiment of the microfluidic device (1) and working principal is shown in FIG. 1A-E. Briefly, it includes two stages namely 1. particle focusing of the cells, and 2. impedance detector using coplanar electrodes.

A representative 2-inlet, 1-outlet microfluidic device as shown in FIG. 1A-F comprises of a particle focusing region (3) (square channel of ˜45 μm with ˜1.2 cm length) and a detection region (4) with a cross-junction (11) and two pairs of coplanar electrodes (20 μm in width and gap) for impedance sensing of cells in native and deformed state. The device layer was fabricated using conventional soft-lithography from SU-8 master mold and bonded on glass substrate with patterned gold electrodes (Au 200 nm and Cr 20 nm). In particular, the microfluidic device of FIG. 1A-F includes a 1) viscoelastic particle focusing region (3) to achieve single stream particle/cell focusing at the channel center (larger dotted box), and 2) a crossflow junction (11) for tuneable cell deformation as a result of compressive (Fc) and shear forces (Fs) using sheath stream (smaller dotted box). Two pairs of coplanar electrodes (8,9) are located before and after the cross junction (11) to perform differential impedance sensing of single cells to electrically quantify cell size, membrane properties and cell deformability at different excitation frequencies.

A exemplary embodiment of the system disclosed herein is illustrated in FIG. 1G, indicating the operational relationship between the microfluidic device (1), transimpedance amplifiers (13), lock-in amplifier (14), fluidic pumps (15), computer system (16) and software programs (17).

The computing system (16) is communicatively coupled to the fluidic pumps (15) to set a flow rate of the sample fluid and sheath fluid through the respective inlets, outlet and channels of the microfluidic device (1). In this regard, the computing system (16) may initiate the perfusion of the fluid sample and sheath fluid through their respective channels by controlling the fluidic pumps (15). However, it will be appreciated that operation of the fluidic pumps may also be performed manually.

Subsequently, one electrode (8b, 9a) in each pair of the microfluidic device (1) is excited at multiple frequencies by the lock-in amplifier (14), whereby the computer system is communicatively coupled to the lock-in amplifier (14) to set the excitation voltage and frequencies to apply to said electrodes (8b, 9a). An impedance response of each cell is obtained by the other electrode (8a, 9b) in each pair and converted via two transimpendance amplifiers (13) into measurements fed into the lock-in amplifier (14). The computer system (16) is communicatively coupled to the lock-in amplifier (14) to receive and process the measurements from the lock-in amplifier, whereby one or more software programs (17) may be used to perform the actions of determining one or more quantitative relationships among impedance responses from one or a combination of the first and second pair of electrodes by quantifying one or more biophysical properties of each cell for profiling of said cell in said fluid sample.

Prior sample and sheath loading into the device, particles/cells were resuspended into viscoelastic medium (Poly(ethylene oxide) in phosphate-buffered saline (1-2.5% w/w PEO (MW 600000) in PBS)) whereas the sheath was pure PEO solution with the same concentration. The sample and sheath fluid were allowed to flow for at least 10 min to stabilize the fluid flow before starting electrical measurement or high-speed imaging. During experiments, the device was mounted on an inverted microscope (Nikon Eclipse Ti). High-speed images (10 kfps, 5 μs) were taken using Phantom V7.3 (AMETEK).

At stage 1, viscoelastic focusing effect (square channel ˜40-50 μm in each side and length ˜1-1.5 cm) was employed to align randomly distributed particles/cells into a single stream.

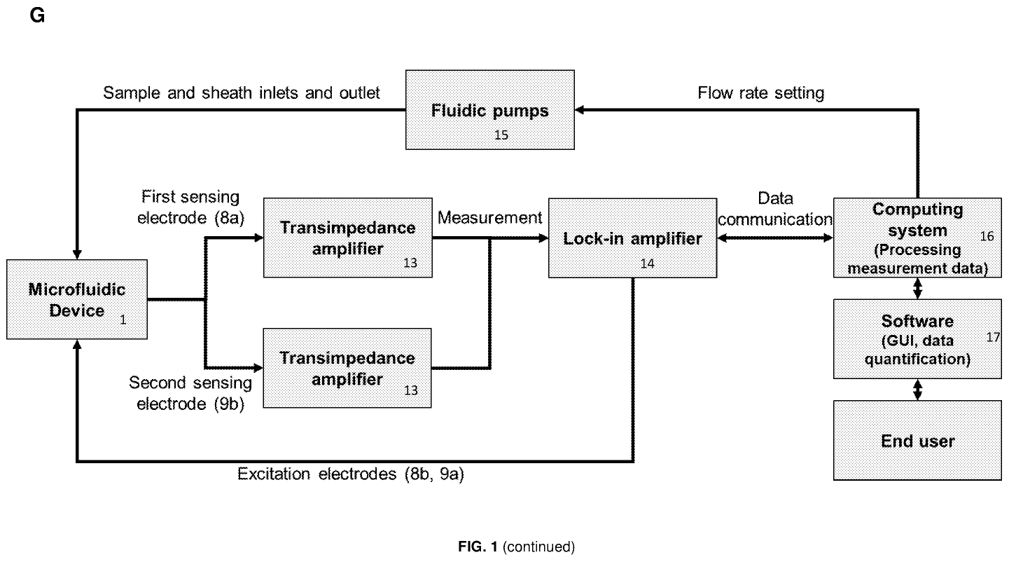

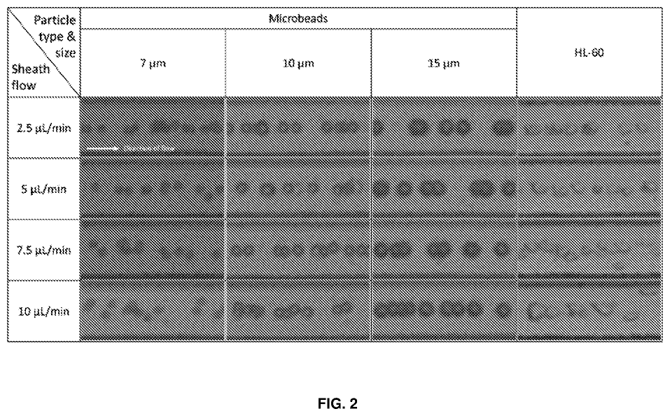

This is an important process for impedance cytometer as variability of particle position can adversely affect impedance readout⁴²⁻⁴⁴. Moreover, similar sized particles/cells aligned in a single stream ensure they will experience the same hydrodynamic forces at the cross junction. FIG. 2 shows successful particle alignment for different sized beads (5, 10 and 15 μm) at sample flow rate ratio ranging from 2.5-10 μL/min.

At stage 2, aligned cells then enter the detection region (square channel with ˜40-50 am width) where a first pair of electrodes (width/gap 20 μm) measures impedance response of cells before deformation (native (|Z|native)).

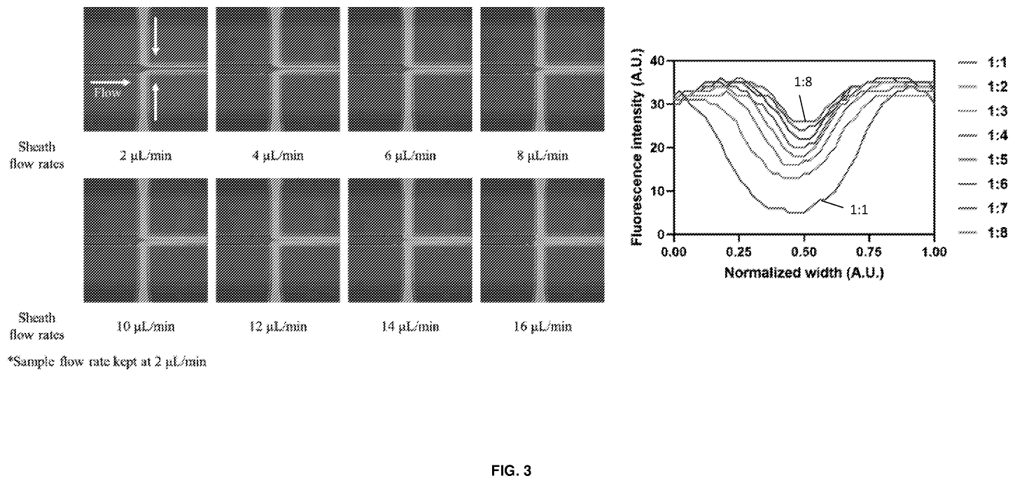

At stage 3, cells are hydrodynamically deformed by sheath stream at the cross junction. FIG. 3 shows different degrees of flow pinching at cross junction due to different flow rate ratios. The use of viscoelastic medium with high polymer (PEO) concentration also helped to achieve large deformation at low flow rate⁴⁵. The cells will retain their deformed shape and flow across a second pair of electrodes where it detects impedance response of cells after deformation (|Z|deform). A force analysis was performed to understand hydrodynamic forces inflict on the cells at the cross junction. Before the cross junction, a cell was flowing in a microchannel at Reynold number of ˜0.01 (Re=ρUDH/μ where ρ is a fluid density of 1000 kg/m3, U is the average sample stream velocity (U=Qᵢₙ/A) of ˜1.6 cm/s (2 μL/min), DH is the hydraulic diameter given by 2WH/(W+H), and μ is a dynamic viscosity of 0.079 Pa·s). At the cross junction, compressive force and shear force are acting on cells^{46,47}.

$${F}_{C} ≅ 0.5 ρ {U}^{2} {C}_{D} {A}_{ρ}$$

where U is average sheath stream velocity (˜8.2 cm/s), CD is the drag coefficient of a sphere (0.47), and Aₚ is the cross-sectional area of a sphere.

$${F}_{S} ≅ \dot{γ} μ ( 4 {πr}^{2} )$$

where \dot{γ} is the shear rate which is estimated as the velocity difference between sample and sheath stream divided by cell diameter⁴⁷.

For 10 μm particle, the compressive force and shear force are approximately 0.13 nN and 0.1 μN, respectively. Comparing with hydropipetting technique using Newtonian fluid at high flow rate (200 μL/min) to induce cell deformation⁴⁷, although the compressive force is smaller, the shear force is comparable and significant cell deformation at low flow rate (10 μL/min) was able to be attained. This low/intermediate flow rate operation is beneficial for impedance cytometry as 1) cell events do not get attenuated by input low-pass filter of the lock-in amplifier (time constant of 5 ρs) and 2) low sampling rate (50 ksample/s) is sufficient to achieve great time/signal resolution while minimizing large data storage and processing time.

By applying excitation signals with multiple frequencies (0.3 MHz, 1.72 MHz, and 12 MHz) using a lock-in amplifier, differential impedance (current) response can be measured and processed on a computer. Quantification of time-series signal provides multiple single cell features namely 1) membrane opacity (ratio of |Z|1.7 MHz to |Z|0.3 MHz), 2) nucleus opacity (ratio of |Z|12 MHz to |Z|0.3 MHz), 3) cell size (|Z|0.3 MHz at native state)⁴⁸ and 4) deformability index from the ratio of native cell signal to deformed cell signal (|Z|Deform/|Z|Native) at 0.3 MHz.

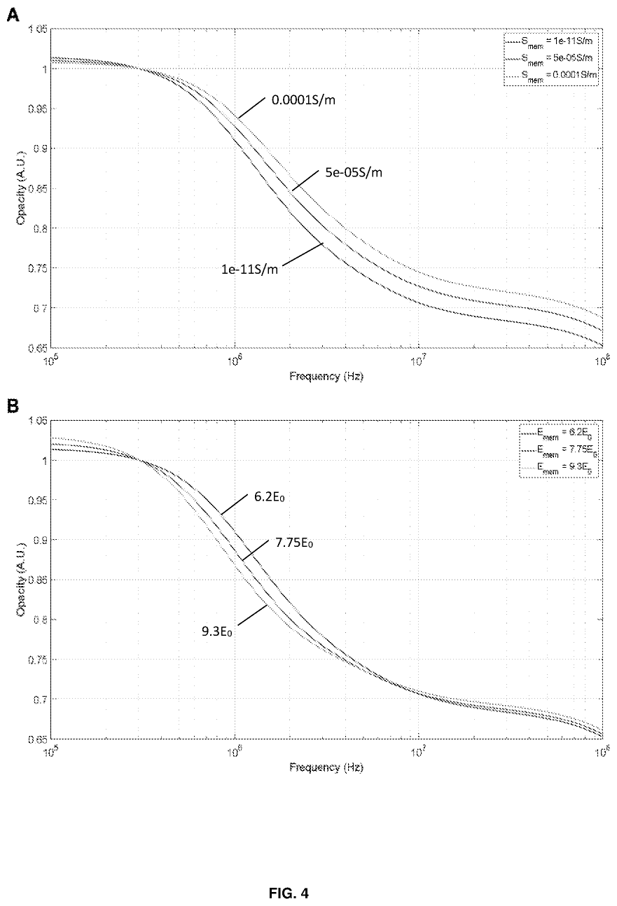

In this regard, to characterize cell membrane properties, the frequency of the excitation signal can be in range of 1-5 MHz as simulation result shows that this can provide information about membrane leakiness (FIG. 4A) and membrane capacitance (FIG. 4B). FIG. 4A illustrates that an increased membrane conductivity (Sₘₑₘ) variation shows that leaky membrane leads to high opacity; FIG. 4B illustrates that membrane capacitance and permittivity variation (Cmem=A*Eₘₑₘ/d, A=surface area, and d=membrane thickness) shows that increased membrane capacitance or surface area (surface morphology of cells e.g., smooth or rough (membrane ruffle, fold)) leads to low opacity.

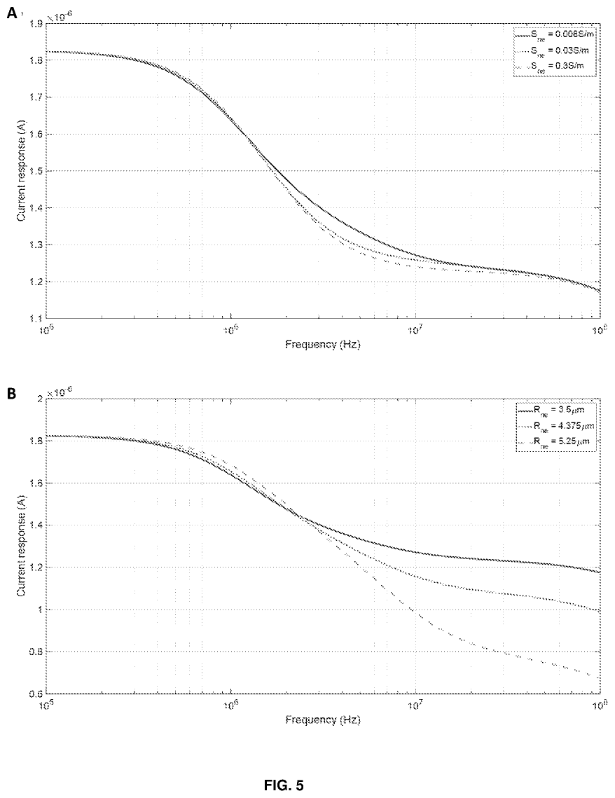

Further, using a frequency of the excitation signal in the range of 5-20 MHz, the nucleus properties such as nucleus leakiness (FIG. 5A) and nucleus size (FIG. 5B) can be probed. FIG. 5A illustrates that an increased nucleus envelope conductivity (Sne) variation shows that leaky membrane leads to low opacity. FIG. 5B illustrates that nucleus size variation shows that increased nucleus size leads to low opacity at high frequency.

Lastly, to measure cell size, a frequency in the range of 0.1-0.5 MHz is employed, as the impedance magnitude correlates to the physical size of cells. By measuring cell size before and after deformation, the electrical deformability index either can be quantified by differencing or normalizing impedance signals between the native and deformed states.

In the following, measurement setup and data presentation of the exemplified embodiments are described.

A differential measurement with two pairs of electrodes was employed to measure single-cell responses. For each pair, one electrode was used to apply an excitation signal of 1 V at two different frequencies (0.3 MHz and 1.72 MHz) or three different frequencies (0.3 MHz, 1.72 MHz, and 12 MHz) by a lock-in amplifier (HF2LI, Zurich instrument) whereas another electrode was used to measure current response from trans-impedance amplifiers (DHPCA-100, FEMTO® or HF2TA, Zurich instruments) with transimpedance gain of 10 kV/A and fed back to lock-in amplifier where differential magnitudes and phase of two pairs at applied frequencies were extracted at a sampling rate of 50 ksample/s. For each experimental condition, measurement was recorded for 3 min. The data processing was done offline to determine single-cell features. Data processing pipeline consists of 1) baseline subtraction to minimize signal drifting due to the influence of low-frequency noise 2) deformed state and native state matching using cross-correlation to determine time differences between deformed state signal and native state signal and k-nearest neighbors search to match deformed state and native state peak magnitudes of the same cells 3) feature normalization to account for setup variability using data from reference beads (15 μm). As described in previous literature⁴³, impedance signal at low frequency of reference beads can be used to determine electrical size of cells (|ZLF| α electric size³). For other features, reference bead features were used for normalizing opacities and electrical deformability index (cell features divided by averaged reference bead feature). Lastly, 2D scatter plots were plotted to illustrate an illustrate biophysical profiles of cell population. All data processing was programmed using MATLAB (MathWorks).

In the following PEO solution preparation in the exemplified embodiments is described.

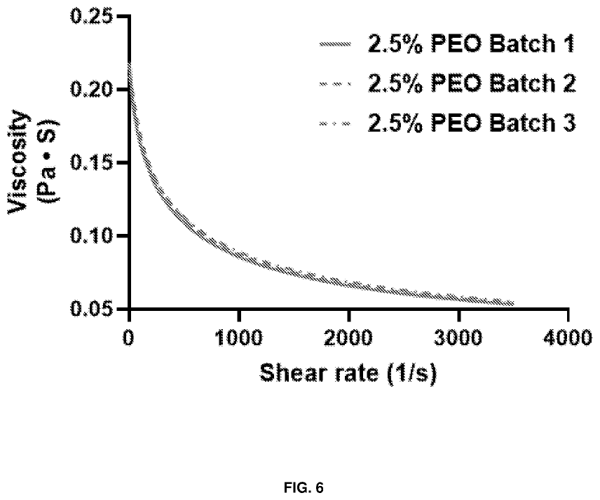

To prepare viscoelastic medium for measurement, PEO (Sigma aldrich) was dissolved in 1×PBS (Lonza) in 250 mL container for 2 days with magnetic stirring (100 rpm) at room temperature. Following that the solution was diluted with 1×PBS to desire concentration for experiments. PEO solution was characterized using Physica MCR501 to acquire relation between viscosity and shear rate (FIG. 6).

In the following, HL60 cell line culture and treatment in the exemplified embodiments are described.

To study mechanical properties of different cell phenotypes, HL-60 (leukemia cell lines) is used as they are proven to be valid model for primary neutrophils⁴⁹. To acquire stiff phenotypes, HL60 were fixed in 4% paraformaldehyde (PFA) for 15 min at room temperature. Then the cells were washed and resuspended in PEO solution for the measurement. For soft phenotype, HL60 was introduced to differentiate into Neutrophil-like cells (Diff) by culturing in well plate with 1.3% of Dimethyl sulfoxide (DMSO) for 4-5 days. After that, the cells were washed and resuspended in PEO for measurement. Untreated HL-60 were used as a control.

In the following, a sample preparation in the exemplified embodiments is described.

To isolate primary neutrophils, a whole blood sample was layered on PolymorphPrep™ at 1:1 ratio. After density centrifugation according to the manufacturer's protocol, the neutrophils were collected and washed with 0.5×PBS. Following that RBC lysis was performed to further remove RBCs. RBC lysis buffer (eBioscience) was added into a neutrophil pellet (1 mL) for 3 min and quenched with 0.5% BSA in PBS. The neutrophils were washed and resuspended in 0.1% BSA in PBS. The cell concentration was determined by Hematocytometer (C-Chip, NanoEnTek). The final concentration for each condition was set to 0.5-1 Mcells/mL.

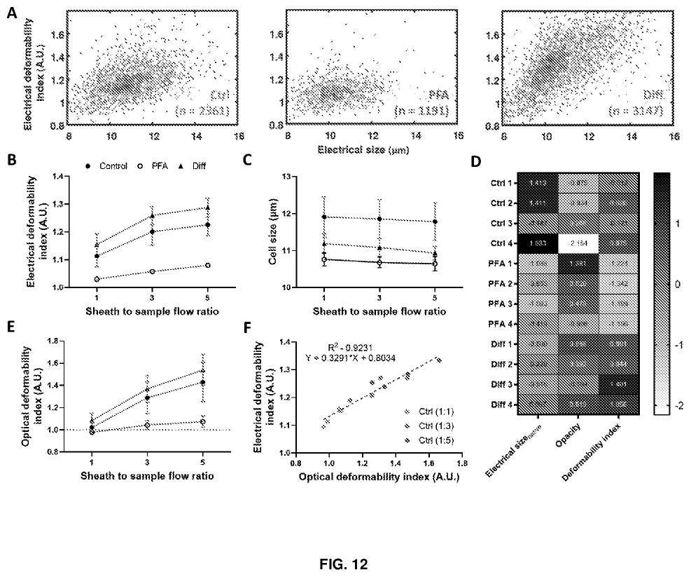

To study the effect of different stimuli on neutrophil biophysical properties, the cells were treated with different chemicals namely 1) N-Formylmethionyl-leucyl-phenylalanine (fMLP, 1 μM) which is a potent chemotactic peptide, 2) Glucose (30 mM) to induce hyperglycemia, and 3) Phorbol Myristate Acetate (PMA, 200 nM) which primes neutrophils to undergo formation of neutrophil extracellular traps (NETosis), as shown in FIG. 12A. For each treatment, 50 μL of cell suspension were incubated with stimulus for 2 hr at 37° C. After 2 hr, 1 mL of PEO solution with 15 μm microbeads was mixed with cell suspension for the measurement. Untreated neutrophils were used as a control.

In addition, to the above treatment steps 1)-3), the cells could be additionally treated with 4) Staurosporine (STS, 1 μM) which causes early apoptosis, and 5) heat shock (HS) treatment (75° C. for 15 min) to induce necrosis, as shown in FIG. 16A.

In the following, flow cytometry analysis of the exemplified embodiments is described.

The staining protocols for leukocyte identification are similar as previously described⁵⁰. The samples were stained for 30 min at 4° C. with FITC-labelled anti-human CD11 b and allophycocyanin (APC)-labelled anti-human CD66b antibodies. Non-specific antibody binding was examined using corresponding isotype negative control antibodies. The cells were washed once prior to flow cytometry analysis. All antibodies were purchased from eBioscience (1:20 dilution) and analyzed using a BD LSR Fortessa flow cytometer (BD Biosciences).

In the following, impedance simulation of the exemplified embodiments is described.

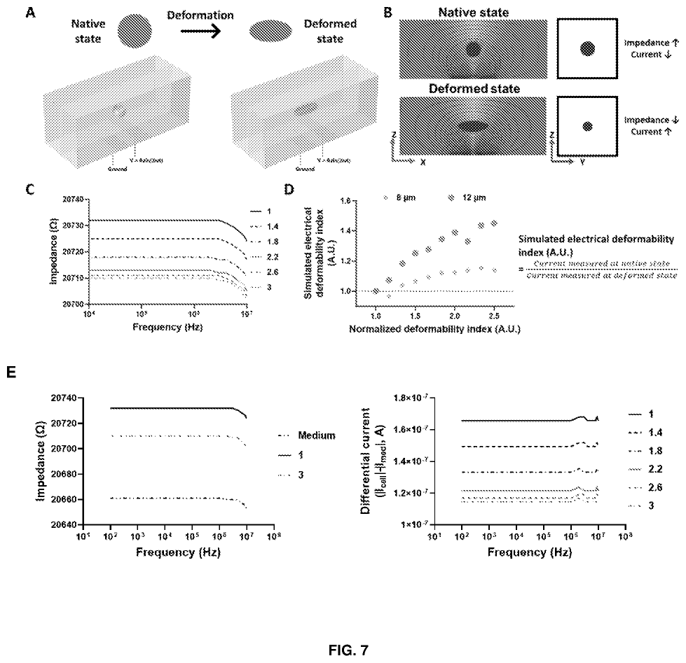

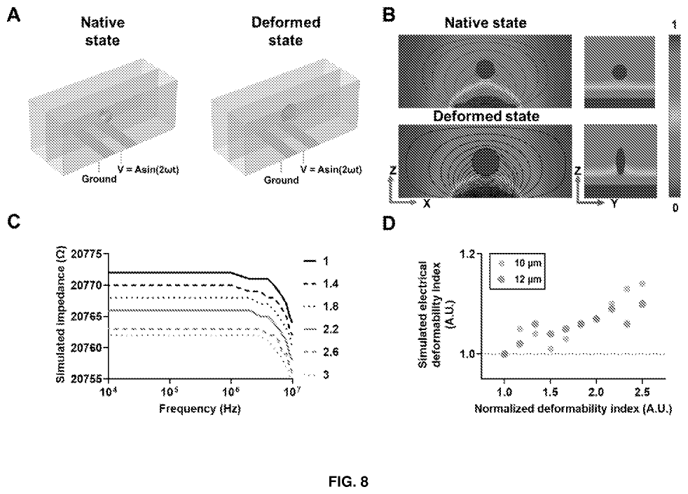

3D numerical simulation was performed to better understand the impedance changes due to different degrees of deformation (FIGS. 7A-D and 8A-D). The 3D numerical simulation of deformed cells in microfluidic channel with coplanar electrodes was setup using the Electric Currents (ec) module in COMSOL Multiphysics 5.0. In the 3D model, an ellipsoid cell or oblate spheroid of volume V_{cell} was located at the center of a segment of microfluidic channel of width w, height h, and depth d (FIGS. 7A and 8A). Three semi-axes of the ellipsoid were a, b, and c in x, y, and z directions, respectively. The channel was filled with medium with electrical conductivity am relative permittivity εm.

Deformed particles were deformed as ellipsoids or oblate spheroids with various deformability indexes (major axis/minor axis >1). Non-deformed particles were modelled as a spheroid (major axis/minor axis=1). For the sake of comparison, both non-deformed and deformed particles were of equal volume.

To study the effects of cell deformation on impedance magnitude, multi-frequency alternating voltage was applied to the right electrode, and the left electrode was grounded. As cell dielectric properties would not significantly affect the investigation of cell deformation-induced impedance change, the cell was assumed non-conductive for simplicity. Deformation index was defined as ratio of a to b (r_ab=a/b), and was varied to study the effects of cell deformation on impedance magnitude. Upon deformation, the cell was assumed to be consistent in volume, namely it was elongated in x direction, and compressed in y and z directions to specific extent.

The governing equation Ohmic conduction was solved to obtain the electric potential distribution:

$$∇ \cdot J = Q \qquad ( 1 )$$

$$J = σ E + jwD + {J}_{e} \qquad ( 2 )$$

$$E = ∇ \cdot V \qquad ( 3 )$$

where J≡J(x,y,t) is the current density (A m⁻²), E≡(x,y,t) is the electric field (V m⁻¹), D D(x,y,t) is the electric displacement field (C m⁻²), Jₑ≡Jₑ(x,y,t) is the external current density (A m⁻²), and V≡V(x,y,t) is the electric potential (V).

Electric insulation boundary condition was applied to the boundaries of the cell and boundaries of the system, except for the electrode regions where external electrical potentials were applied.

$$n \cdot J = 0 \qquad ( 4 )$$

where n is the normal vector perpendicular to the boundary pointing out of the computational domain.

The left electrode was grounded.

$$V = {V}_{L} = 0 \qquad ( 5 )$$

Alternating voltage of magnitude of V_{L} was applied to the right electrode.

$$V = {V}_{R} \qquad ( 6 )$$

The governing equations 1-3 were solved with the boundary conditions 4-6 using finite element methods in a frequency domain. Impedance of the system was evaluated based on the admittance at the electrode. Impedance of a cell-free microchannel was subtracted from the impedance of cell-loaded microchannels to obtain the impedance of a single cell.

TABLE 1
Impedance Modelling parameters

| | Nota- | |
|---|---|---|
| Parameter | tion | Value |
| Cell volume | V_{Cell} | 4/3 · πr³ [μm³] |
| Channel width | w | 100 [μm] |
| Channel height | h | 40 [μm] |
| Channel depth | d | 40 [μm] |
| Electrical conductivity of medium | σₘ | 1.6 [S/m] |
| Relative permittivity of medium | εₘ | 80 |
| Deformation index | r_ab | 1-3 |
| Semi-axis of cell in x direction | a | V_{Cell}\hat{ }(⅓) *r_ab\hat{ }(⅔) |
| Semi-axis of cell in y direction | b | V_{Cell}\hat{ }(⅓) * r_ab\hat{ }(−⅓) |
| Semi-axis of cell in z direction | c | V_{Cell}\hat{ }(⅓) * r_ab\hat{ }(−⅓) |
| Voltage applied on the left | V_{L} | 0 |
| electrode (ground, sensing) | | |
| Voltage applied on the right | V_{R} | 1 [V] |
| electrode (excitation) | | |

In the following, image processing is being described.

To obtain consistent results and avoid untransparent electrodes areas, the program first crops the video (resolution of 512×64 pixels) to 110×64 pixels from either left (before deformation) or right (after deformation) side and saves them as separate clips. Then the background subtraction was performed followed by image thresholding to segment flowing cells. Next, a fit ellipse function was applied. This function models segmented cells or a group of connected pixels into ellipse shape and returns its information including centroid position, size by area, perimeter, major and minor axis lengths, and tilting angle. All above information was stored as a .csv file for further verification or processing. The optical deformability index is calculated by major axis/minor axis. The average and standard deviation of the deformability index is obtained for correlation analysis and visualization.

To determine cell number, the program uses a simple tracking and labelling function. First, a size exclusive selection was performed. Particles with a size of less than 30 pixels are recognized as impurities, and the frames containing these particles are removed. Next, the frame number and centroid X (cX) value was used to cluster cell frames. If in two adjacent frames, the cell moves in the correct direction (cX₂−cX₁ is positive) and that the absolute difference between the centroid X values is less than a threshold, these two cells are then assigned the same label.

In the following, results and discussion of the exemplified embodiments are being described.

### Example 1: Tunable Cell Deformation for Impedance-Based Quantification

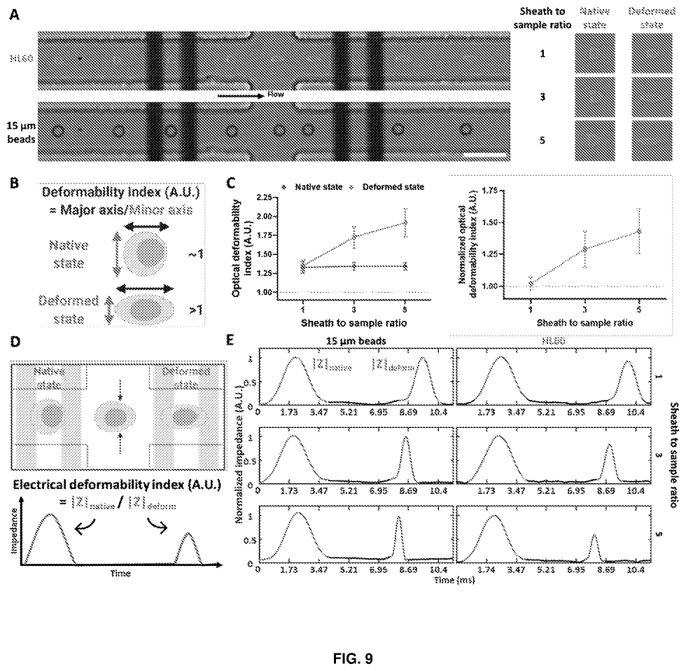

The proposed detection scheme was first validated with HL60 cells and 15 μm polystyrene beads. FIG. 9A shows time sequence images of HL60 and 15 μm beads flowing through the detection region at sample flow of 2 μL/min and sheath flow rate of 10 μL/min. It was observed the cells deformed noticeably after the cross junction due to hydrodynamic pinching by the sheath flow whereas microbead did not exhibit any noticeable deformation. The optical deformability index was defined as a ratio between major axis and minor axis (FIG. 9B).

As expected, the higher sheath flow rate led to more cell deformation as evident in the snapshot images as well as quantification of optical deformability index (FIG. 9B-C). Analysis of impedance signal was also performed in FIG. 9D. By employing differential current sensing, the first measurement is taken before cell deformation which represents the native state signal((|Z|native), and the second measurement is taken after the cross junction to represent a deformed state signal with a lower magnitude (|Z|deform). The electrical deformability index is defined as the ratio between impedance signal before deformation (|Z|native) and impedance signal after deformation (|Z|deform).

FIG. 9E shows a representative signal for 15 μm beads and HL60 at different flow rate ratios. As the flow rate ratio increased, signal magnitudes (|Z|deform) of HL60 decreased while bead signal magnitudes (|Z|deform) of beads had negligible changes. These results demonstrate the use of impedance signature to quantify cell deformability.

### Example 2: Impedance Simulation of Particles with Different Shapes

3D numerical simulation was performed to better understand the impedance changes due to different degrees of particle/cell deformation. Non-deformed particles were model as a spheroid (major axis/minor axis=1).

Initially, an impedance simulation was performed on ellipsoids as the deformed particles (FIG. 7A-D). Simulation results revealed that impedance magnitude decreased as the deformability index increased in a non-linear manner (FIG. 7C). It was hypothesized that deformed objects have smaller apparent size (cross-sectional view) which permits more current flow leading to lower impedance as compared to non-deformed objects (native state). To account for the actual measurement scenario, simulated impedance magnitude at 0.3 MHz was converted into simulated electrical deformability index (current response of native state divided by current response of deformed state, FIG. 7E), and it was found that simulated electrical deformability index correlated well with normalized deformability index (FIG. 7D). It was observed that particle size also influenced simulated electrical deformability index as relative size changes of small particles may render smaller changes in deformed state impedance resulting in smaller changes in electrical deformability index. Overall, these results suggest the feasibility of using differential impedance signals to electrically quantify cell deformability.

In addition, due to uncertainty in the actual shape of the deformed particles, an impedance simulation was also performed on oblate spheroids as the deformed particles (FIG. 8A-D) and the results showed that the simulated electrical deformability index was correlated to particle shapes with varying optical deformability index. This suggests a robust sensor mechanism to assess the deformability of differently shaped particles.

### Example 3: Biophysical Profiling of HL-60 Cells

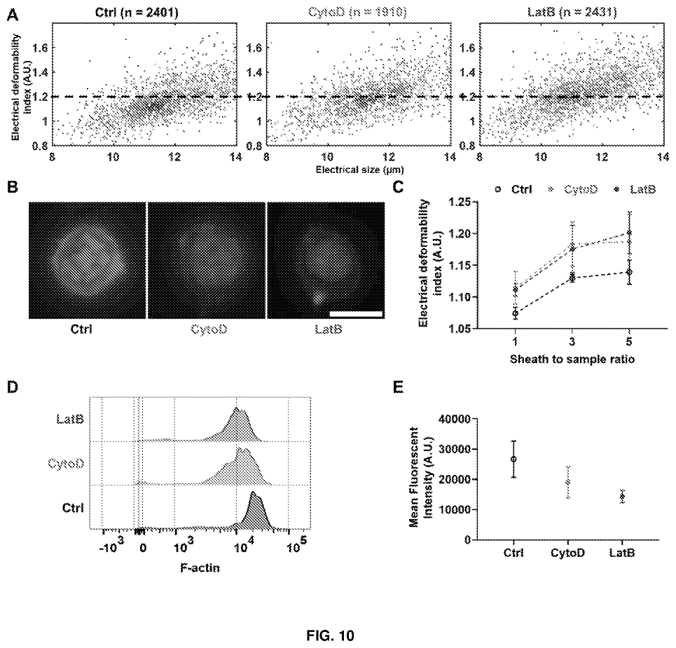

The effect of cytoskeleton disruptors, such as cytochalasin D (CytoD) and latrunculin B (LatB), on HL-60 deformability, was studied (FIG. 10 A-E). Impedance measurement reveals that treated HL-60 were more deformable as compared to a control as evident in higher electrical deformability index. Consistent with previous work^{29,51}, immunostaining images and flow cytometric analysis showed that treated HL-60 exhibited decreased F-actin expression as compared to control indicating softer phenotype (FIG. 10 B, D, E).

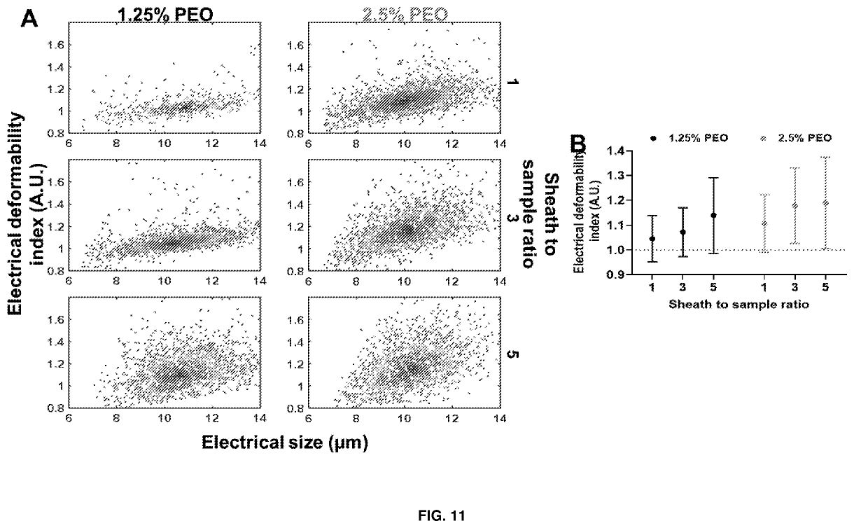

The effect of different concentrations of PEO in the sample and sheath fluid, on HL-60 deformability, was studied (FIG. 11A-B). It was found that lower PEO concentration induced lower cell deformation. Higher PEO concentrations (>2.5%) can further increase the shear forces for greater cell deformation but may lead to device debonding/leakage due to higher pressures. Shear rate modulation by adjusting PEO concentration can thus be beneficial for studying mechanical properties of different cellular structures²⁹.

Application of the microfluidic device for mechanical characterization of different HL60 phenotypes was next determined, namely 1. Untreated control (ctrl), 2. Paraformaldehyde fixed (PFA) and 3. Differentiated into neutrophil-like cells (Diff). FIG. 12A shows mechanical profiles of Ctrl, PFA and Diff-HL60. The electrical deformability index of the PFA-HL60 were lower than untreated control as PFA crosslinked cell membrane led to higher cell stiffness⁵² (FIG. 12B). PFA treatment also resulted in smaller cell size due to cell shrinkage (FIG. 12C). For the differentiated HL-60, an increase in electrical deformability index and a decrease in cell size was observed which could be attributed to the differentiation process (FIGS. 12B and C). These findings were also consistent with the measurement using a real-time deformability cytometer⁵². FIG. 12D shows the heatmap representing individual measurements from 4 independent experiments indicating consistent trends across different experiments. Measurement of optical deformability index using a high-speed camera also revealed similar trends for different phenotypes (FIG. 12E). Lastly, a correlation analysis was performed between electrical and optical measurement which found that the electrical deformability index was in good agreement with the optical formability index (FIG. 12F), which clearly indicates the utility of impedance signal to assess cell mechanical deformation for multi-parametric single-cell measurement.

### Example 4: Multi-Parametric Biophysical Profiling of Primary Neutrophils

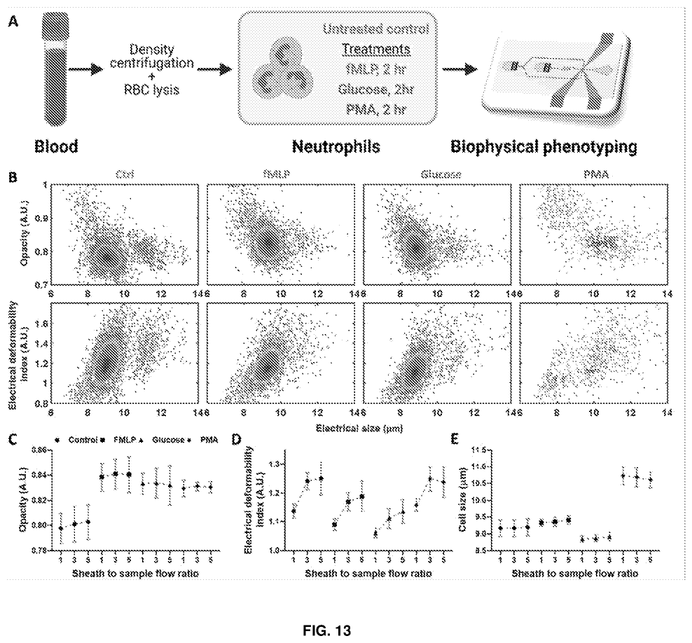

As a proof-of-concept for clinical testing, the microfluidic platform disclosed herein was utilized to electrically characterize biophysical properties of primary neutrophils treated with different chemicals. As shown in FIG. 13A, neutrophils were isolated from whole blood using density centrifugation (Polymorphprep™) followed by red blood cell lysis. The isolated neutrophils were activated for 2 hr with 1) N-Formylmethionine-leucyl-phenylalanine (fMLP, 1 μM) which is potent chemotactic peptide, 2) Glucose (30 mM) to imitate elevated glucose condition in Diabetes Mellitus, and 3) Phorbol Myristate Acetate (PMA, 200 nM) which prompts neutrophils to undergo neutrophil extracellular trap formation (NETosis).

Deformability profiles revealed that fMLP and glucose treated neutrophils exhibited stiffer cell properties as compared to untreated control and NETosis population. The decreased deformability index for activated phenotypes (fMLP and glucose) was likely attributed to an increased F-actin as observed in fMLP treated neutrophils⁵⁴. In terms of membrane properties (FIG. 13B, C), elevated opacity for all treated conditions as compared to the untreated control was observed. The increased opacity for treated conditions was probably due to compromised membrane integrity (high current penetration) resulting in high opacity⁴². Interestingly, the impedance profile of PMA treated neutrophils revealed two distinct clusters 1) small cell size with high opacity and 2) large cell size with low opacity. The first cluster was probably dead neutrophils with compromised membrane whereas the latter was neutrophils undergoing NETosis which become larger in size⁴¹. To simplify analysis for PMA, the dead cell cluster was excluded in the subsequent analysis.

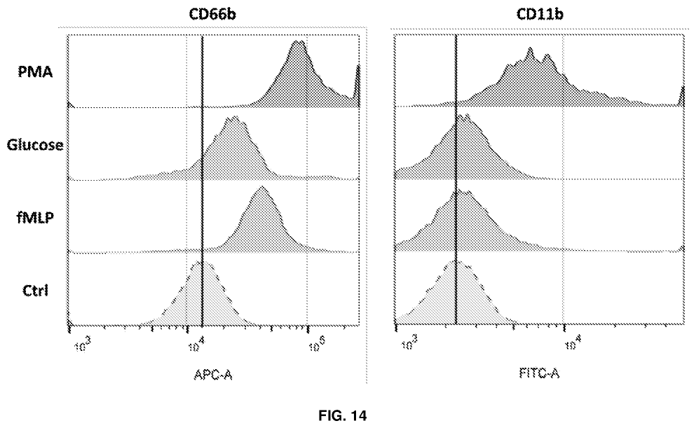

Flow cytometric analysis was performed to confirm the activation status of neutrophils based on CD66b and CD11 b which are common neutrophil activation markers⁵⁵, and found that fMLP, glucose and PMA treatment led to neutrophil activation as evident in elevated expressions of CD66b and CD11 b (FIG. 14). As shown in FIG. 13B, D, deformability profiles revealed that fMLP and glucose treated neutrophils exhibited stiffer properties as compared to untreated control and NETosis population. The decreased deformability index for activated phenotypes (fMLP and glucose) was likely due to an increased F-actin as observed in fMLP treated neutrophils⁵⁴.

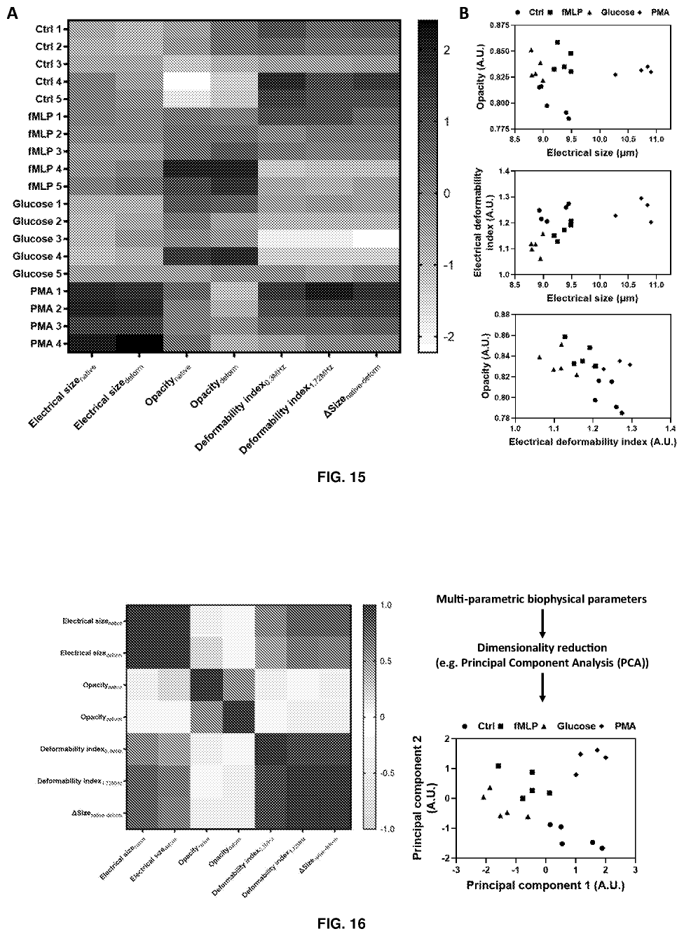

For the cell size (FIG. 13B, E), it was found that fMLP and PMA treated neutrophils were larger than control while glucose treated neutrophils were smaller. FIG. 15A shows a heatmap of biophysical features indicating similar trends were observed in different donors. Multifrequency impedance measurement can extract features namely 1) electrical size of deformed state, 2) opacity of deformed state 3) electrical deformability index from 1.72 MHz and 4) size difference between native and deformed state. Though these parameters followed similar trends as the main parameters, they may possess different biophysical meanings which may provide a distinct signal in other applications.

By pairing different main biophysical parameters (FIG. 15B), different phenotypic changes in 2D scatter plots can be observed which can be utilized to gate certain phenotypes for analysis. With multidimensional biophysical features, one can apply machine learning techniques to enable better cell classification (FIG. 16). It should be noted that increasing number of frequencies of excitation signal could result in additional features for analysis. For example, adding excitation signal with frequency above at 10 MHz would enable interrogation of nucleus properties as well as electrical deformability index at high frequency which may provide interesting insights regarding nucleus deformation. Overall, these results demonstrate the feasibility of multi-parametric biophysical characterization of neutrophils which potentially translates to the study of neutrophils in a disease context.

### Example 5: Multi-Parametric Biophysical Profiling of Primary Neutrophils

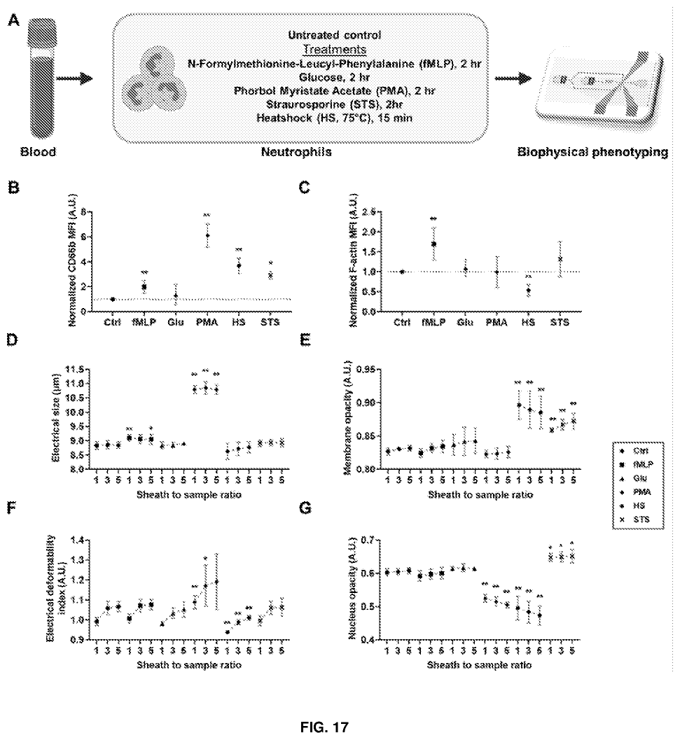

As a proof-of-concept for clinical testing, the microfluidic platform disclosed herein was utilized to electrically characterize biophysical properties of primary neutrophils treated with different chemicals. Neutrophils were isolated from whole blood using density centrifugation (Polymorphprep™) followed by red blood cell lysis (FIG. 17A). The purified neutrophils were then treated (2 hr) with 1) N-Formylmethionine-leucyl-phenylalanine (fMLP, 1 μM) which is a potent chemotactic peptide, 2) glucose (30 mM) to induce hyperglycemia, 3) Phorbol Myristate Acetate (PMA, 200 nM) which primes neutrophils to undergo formation of neutrophil extracellular traps (NETosis), 4) Staurosporine (STS, 1 μM) which causes early apoptosis, and 5) heat shock (HS) treatment (75° C. for 15 min) to induce necrosis.

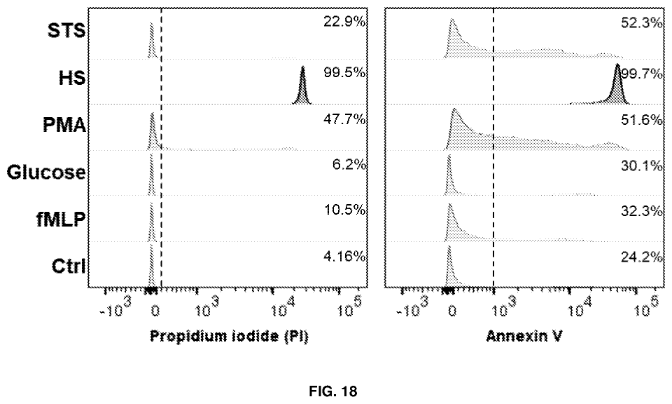

Flow cytometric analysis was first performed and showed that all treated neutrophils (except glucose) exhibited a significant increase in CD66b expression which indicates elevated cell activation³⁸ (FIG. 17B). fMLP-treated neutrophils had the highest F-actin expression while HS neutrophils showed a significant reduction (FIG. 17C). To confirm necrosis and early apoptosis events, Annexin V/Propidium iodide (PI) staining was performed, and increased Annexin V expression and high Annexin V/PI expressions for STS and HS was observed, respectively (FIG. 18).

Multi-parametric biophysical profiling of neutrophils was subsequently performed using the developed impedance cytometer device at different flow rate ratios (FIG. 17D-G). It should be noted that unlike cell deformability, significant changes in cell size and membrane/nucleus opacity were not observed with different flow rate conditions, thus indicating that these biophysical properties are independent of cell deformation. Neutrophil size increased with fMLP treatment, and PMA-treated neutrophils exhibited the largest increase in cell size (FIG. 17D). For membrane properties, a significant increase in membrane opacity was observed for HS- and STS-treated neutrophils as compared to control (FIG. 17E). This is likely due to the compromised membrane integrity (“more leaky”)⁵⁶ with high PI expression (FIG. 18) which led to more current flow (lower |Z|0.3 MHz) and thus higher opacity (|Z|1.7 MHz/|Z|0.3 MHz)⁴².

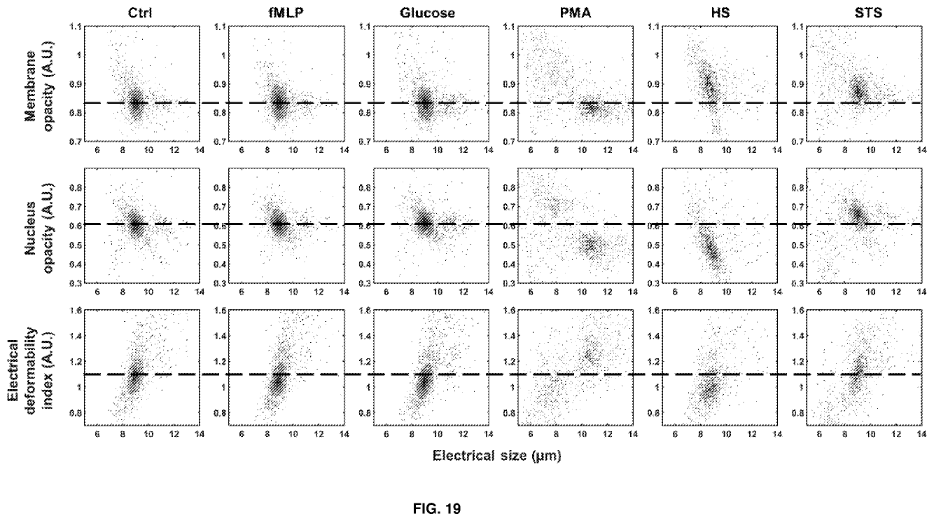

It was observed that the impedance profile of PMA-treated neutrophils revealed two distinct cell clusters comprising of 1) small cell size with high opacity, and 2) large cell size with low membrane opacity neutrophils (FIG. 19). The first cell cluster probably consists of dying neutrophils (with completed NETosis) with ruptured cell membrane as seen in elevated PI, while the 2nd population may be neutrophils undergoing NETosis as cell size increase is associated in early NETosis events (nucleus degradation)⁴⁶.

For electrical cell deformability characterization, PMA-treated neutrophils became more deformable, while HS-treated neutrophils were stiffer as compared to untreated neutrophils (FIG. 17F). There were also two distinct cell populations of different size and deformability (FIG. 19) for PMA-treated neutrophils, possibly due to neutrophils undergoing different stages of NETosis. For example, the smaller and stiffer neutrophils with higher membrane opacities may be indicative of cell death after NETosis. The larger neutrophils with lower opacities and higher cell deformability may be in early NETosis phase as the cells undergo nuclear changes (e.g. chromatin decondensation) but has not yet initiated the membrane rupture process. Although fMLP-treated neutrophils showed increased F-actin expression due to actin polymerization during chemotaxis as reported previously⁵⁴, there were negligible changes in electrical deformability index. This warrants further investigations but might be related to treatment duration or confounding changes in cell size⁵⁷ (FIG. 17D). HS neutrophils showed reduced deformability index despite having lower F-actin expression, and this could be due to thermal treatment which disrupts actin distribution⁵⁸ (FIG. 17C).

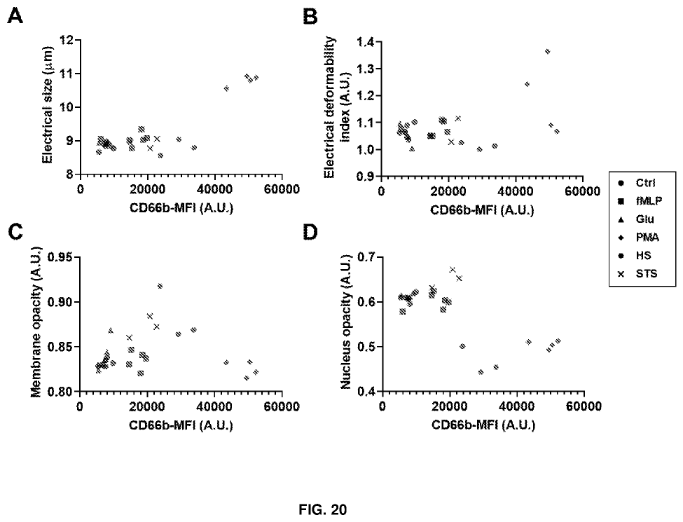

For nucleus properties, a significant decrease in nucleus opacities (|Z|12 MHz/|Z|0.3 MHz) was observed for PMA- and HS-treated neutrophils, while STS-treated neutrophils had higher nucleus opacity (FIG. 17G). According to the simulation results, the lower nucleus opacities are attributed to increased nucleus size due to nucleus swelling for PMA and increased conductivity of nucleus envelope due to increased nucleus permeability for HS⁵⁹. STS-treated neutrophils had slightly higher nucleus opacity which suggests an intact nucleus in early apoptosis event as reported by others⁵⁶. To better visualize the associated biophysical changes, the results were presented in 2D impedance plots which highlights distinct biophysical changes with different treatments (FIG. 19). Lastly, a correlation analysis was performed between neutrophil activation marker (CD66b) and various biophysical parameters (FIG. 20). An increase in CD66b expression with cell size for fMLP- and PMA-treated neutrophils was observed, but not STS and HS treatment probably due to different modes of action by the stimulus. Taken together, these results clearly showcase the potential and superiority of the various embodiments disclosed herein to measure multiple biophysical cell markers to study neutrophil health and functions.

### Example 6: UMAP Analysis of Neutrophil Biophysical Markers

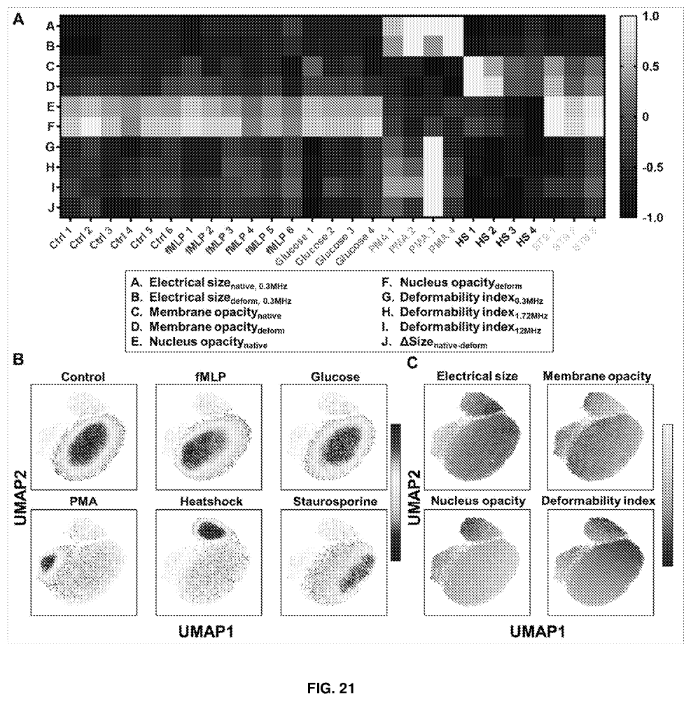

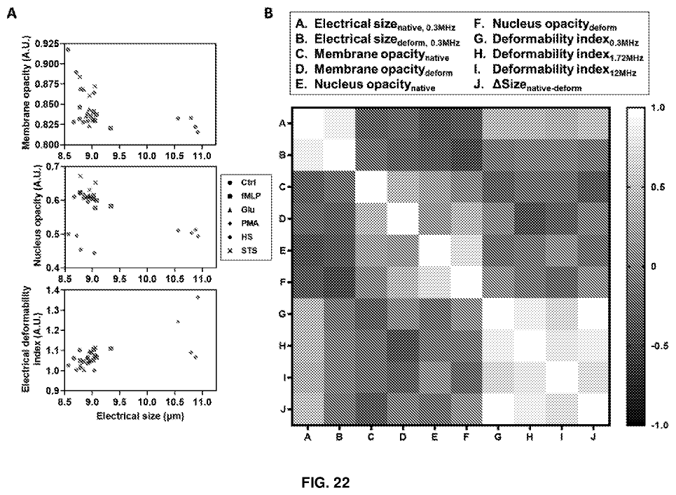

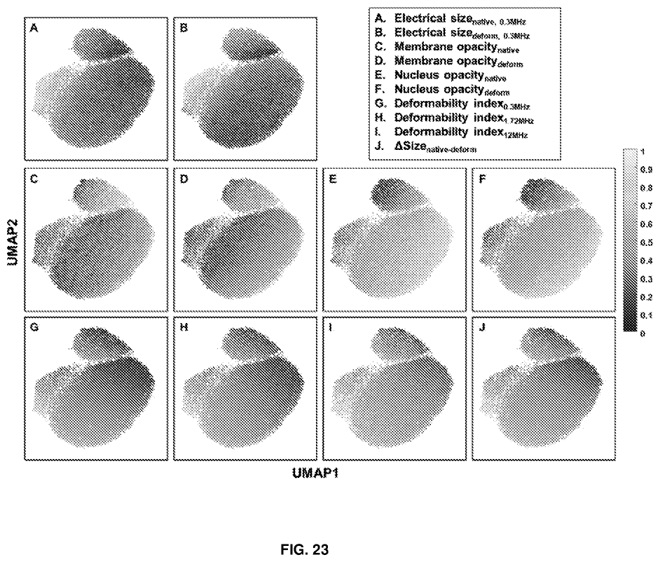

To better understand the influence and interdependency of the measured single-cell features, a heatmap analysis was performed on the biophysical parameters for all the donor samples and their treated conditions (FIG. 21A). These markers were first analysed in 2D scatter plots to assess if distinct mechano-phenotypes exist for each stimulus or in both magnitude and direction (FIG. 22A).

In addition to the four cell parameters (native cell size, native membrane opacity, native nucleus opacity and electrical deformability index (4 parameters)), the multi-frequency impedance measurements also allowed more (up to 10 in total) cellular features to be extracted by considering deformed cell sizes (1 parameter), opacities (2 parameters) and electrical deformability index at different frequencies (3 parameters). Based on this analysis, it is evident that the four main biophysical markers were considered independent cell characteristics (FIG. 22B). Consistent results were also observed among donors, which indicates that the microfluidic platform disclosed herein is robust to detect multi-parametric single-cell biophysical behavior. Although the newly extracted parameters seemed to be closely associated with the main biophysical markers for neutrophils, these features may be useful in other cell types (e.g. stem cells) where cells are known to be highly heterogeneous, and for machine learning or cell classification purposes.

To facilitate visualization of the neutrophil multi-dimensional data, Uniform Manifold Approximation and Projection for Dimension Reduction (UMAP) to reduce biophysical features into two dimensions. Localized density maps of different phenotypes indicated distinct cell distribution and patterns (FIG. 21B, 23). Magnitude analysis on the four major features (size, deformability, membrane opacity and nucleus opacity) was also performed to understand their contribution to UMAP and distinct changes in terms of biophysical signatures (FIG. 21C). Since PMA, HS and STS are potent stimuli that lead to neutrophil death through different mechanisms, these conditions exhibited strong biophysical changes as part of the functional responses⁶⁰⁻⁶². These results also showed that fMLP- and glucose-treated (a mild stimulus) neutrophils showed smaller shifts in different directions, thus suggesting that multi-parametric cell phenotyping could help in better phenotyping segregation. Overall, these results demonstrate the feasibility of multi-parametric biophysical characterization of single neutrophils which can be readily applied for other cell types (e.g. stem cells⁶³) and clinical diagnostics^{27,64}.

### Example 7: COVID Recovered Patients' Neutrophil Study

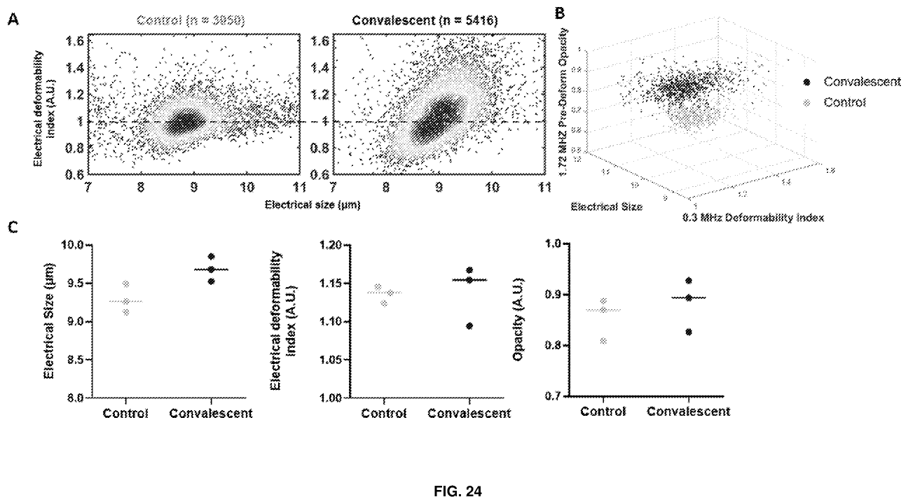

Coronavirus disease (COVID) is rapidly spreading all over the world since its outbreak in 2019. Recent study reported that the biophysical phenotypes for COVID patients and convalescents are changed⁶⁵. To verify this finding, the isolated neutrophils were tested (density centrifugation using Polymorphprep™) from both convalescent and healthy donor with the method and microfluidic device disclosed herein. The results showed that COVID recovered patients' neutrophil were bigger and had higher electrical deformability indices (FIG. 24), which were in good agreement with earlier findings.

### Example 8: Cancer Cells Multi-Parametric Biophysical Profiling

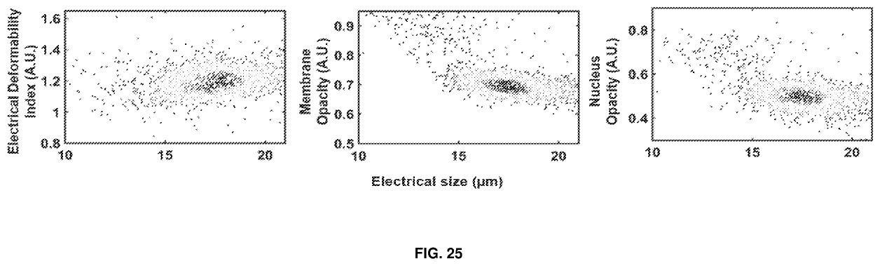

The utility of the method and microfluidic device disclosed herein was further investigated in profiling the biophysical parameters for mammalian adherent cells. MCF-7 cells (breast cancer cell line) were trypsinized and were homogeneously resuspended in the viscoelastic medium prior to the mechano-phenotyping process where the capability of measuring the size, deformability, membrane opacity and nucleus opacity for MCF-7 cells was shown (FIG. 25). This result demonstrated this device's capability in profiling adherent cells biophysical properties and revealed a broader scope for possible future applications (eg, mesenchymal stem cells, fibroblast).

### Example 9: Electro-Mechano-Phenotyping of Single Leukocytes for Label-Free Immunoprofiling in Type 2 Diabetes Mellitus

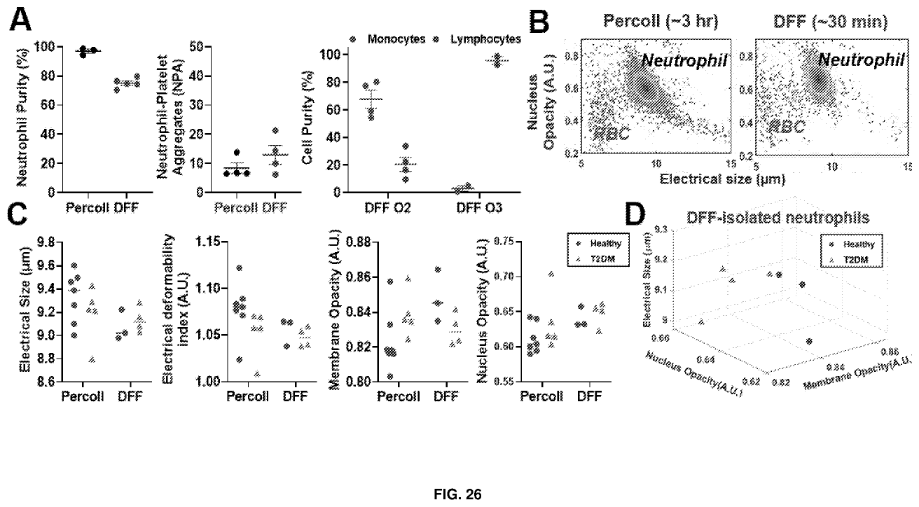

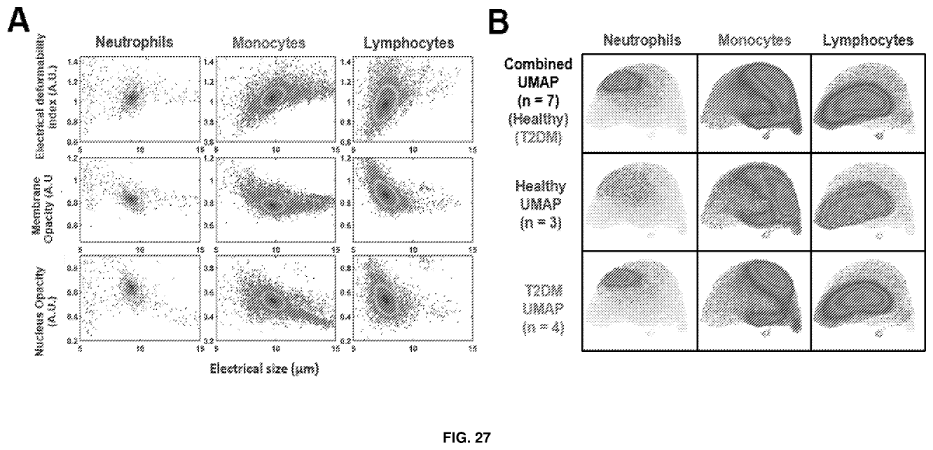

Conventional density centrifugation (Percoll) and microfluidics Dean Flow Fractionation (DFF) was used to isolate different leukocyte subtypes (FIG. 26 A, B) for single cell electro-mechano-phenotyping using the method and microfluidic device disclosed herein. The results showed that neutrophils isolated from healthy and type 2 diabetes mellitus (T2DM) subjects were able to be distinguished based on biophysical features (FIG. 26 C, D). The electro-mechano-phenotypes for monocytes and lymphocytes were then further mapped (FIG. 27A). Distinct differences were observed between healthy and T2DM samples for all leukocyte subtypes based on Uniform Manifold Approximation and Projection (UMAP) analysis of 4 biophysical parameters (FIG. 27B), suggesting its potential for label-free immunoprofiling.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

1. Rosales, C. Neutrophil: A Cell with Many Roles in Inflammation or Several Cell Types?Frontiers in Physiology 9, (2018).
2. Mendonça, M. A., Cunha, F. Q., Murta, E. F. & Tavares-Murta, B. M. Failure of neutrophil chemotactic function in breast cancer patients treated with chemotherapy. Cancer chemotherapy and pharmacology 57, 663-670, doi:10.1007/s00280-005-0086-4 (2006).
3. Granot, Z. & Jablonska, J. Distinct Functions of Neutrophil in Cancer and Its Regulation. Mediators of inflammation 2015, 701067-701067, doi:10.1155/2015/701067 (2015).
4. Alba-Loureiro, T. C. et al. Neutrophil function and metabolism in individuals with diabetes mellitus. Brazilian journal of medical and biological research=Revista brasileira de pesquisas medicas e biologicas 40, 1037-1044, doi:10.1590/s0100-879x2006005000143 (2007).
5. Hughes, M. J., McGettrick, H. M. & Sapey, E. Shared mechanisms of multimorbidity in COPD, atherosclerosis and type-2 diabetes: the neutrophil as a potential inflammatory target. European Respiratory Review 29, 190102, doi:10.1183/16000617.0102-2019 (2020).
6. Di Carlo, D. A Mechanical Biomarker of Cell State in Medicine. Jala 17, 32-42, (2012).
7. Kang, N., Guo, Q., Islamzada, E., Ma, H. & Scott, M. D. Microfluidic determination of lymphocyte vascular deformability: effects of intracellular complexity and early immune activation. Integr. Biol. 10, 207-217, (2018).
8. Crawford, K. et al. Rapid Biophysical Analysis of Host Immune Cell Variations Associated with Sepsis. Am. J. Respir. Crit. Care Med. 198, 280-282, (2018).
9. Petchakup, C., Li, K. & Hou, H. Advances in Single Cell Impedance Cytometry for Biomedical Applications. Micromachines 8, 87, (2017).
10. Hassan, U., Watkins, N. N., Reddy Jr, B., Damhorst, G. & Bashir, R. Microfluidic differential immunocapture biochip for specific leukocyte counting. Nat. Protocols 11, 714-726, (2016).
11. Simon, P., Frankowski, M., Bock, N. & Neukammer, J. Label-free whole blood cell differentiation based on multiple frequency AC impedance and light scattering analysis in a micro flow cytometer. Lab on a Chip 16, 2326-2338, (2016).
12. Song, H. et al. A microfluidic impedance flow cytometer for identification of differentiation state of stem cells. Lab on a Chip 13, 2300-2310, (2013).
13. Yang, D., Zhou, Y., Zhou, Y., Han, J. & Ai, Y. Biophysical phenotyping of single cells using a differential multiconstriction microfluidic device with self-aligned 3D electrodes. Biosensors and Bioelectronics 133, 16-23, (2019).
14. Zhou, Y. et al. Characterizing Deformability and Electrical Impedance of Cancer Cells in a Microfluidic Device. Analytical Chemistry 90, 912-919, (2018).
15. Chen, J. et al. Classification of cell types using a microfluidic device for mechanical and electrical measurement on single cells. Lab on a Chip 11, 3174-3181, (2011).
16. Zhao, Y. et al. Development of microfluidic impedance cytometry enabling the quantification of specific membrane capacitance and cytoplasm conductivity from 100,000 single cells.

Biosensors & bioelectronics 111, 138-143, (2018).
17. Lakschevitz, F. S. et al. Identification of neutrophil surface marker changes in health and inflammation using high-throughput screening flow cytometry. Experimental Cell Research 342, 200-209, (2016).
18. Perdomo, J. et al. Neutrophil activation and NETosis are the major drivers of thrombosis in heparin-induced thrombocytopenia. Nature Communications 10, 1322, (2019).
19. Sun, T. & Morgan, H. Single-cell microfluidic impedance cytometry: a review. Microfluidics and Nanofluidics 8, 423-443, (2010).
20. Chen, J. et al. Microfluidic Impedance Flow Cytometry Enabling High-Throughput Single-Cell Electrical Property Characterization. International Journal of Molecular Sciences 16, 9804, (2015).
21. Honrado, C., Bisegna, P., Swami, N. S. & Caselli, F. Single-cell microfluidic impedance cytometry: from raw signals to cell phenotypes using data analytics. Lab on a Chip 21, 22-54, (2021).
22. Pethig, R. et al. Dielectrophoretic studies of the activation of human T lymphocytes using a newly developed cell profiling system. Electrophoresis 23, 2057-2063, (2002).
23. Huang, Y., Wang, X.-B., Gascoyne, P. R. C. & Becker, F. F. Membrane dielectric responses of human T-lymphocytes following mitogenic stimulation. Biochimica et Biophysica Acta (BBA)—Biomembranes 1417, 51-62, (1999).
24. Watkins, N. N. et al. Microfluidic CD4<sup>+</sup> and CD8<sup>+</sup>T Lymphocyte Counters for Point-of-Care HIV Diagnostics Using Whole Blood. Science Translational Medicine 5, 214ra170-214ra170, (2013).
25. Rollo, E. et al. Label-free identification of activated T lymphocytes through tridimensional microsensors on chip. Biosensors and Bioelectronics 94, 193-199, (2017).
26. Han, P. et al. Continuous Label-Free Electronic Discrimination of T Cells by Activation State. ACS Nano 14, 8646-8657, (2020).
27. Gossett, D. R. et al. Hydrodynamic stretching of single cells for large population mechanical phenotyping. Proceedings of the National Academy of Sciences 109, 7630, (2012).
28. Deng, Y. et al. Inertial Microfluidic Cell Stretcher (iMCS): Fully Automated, High-Throughput, and Near Real-Time Cell Mechanotyping. Small 13, 1700705, (2017).
29. Urbanska, M. et al. A comparison of microfluidic methods for high-throughput cell deformability measurements. Nat Methods 17, 587-593, (2020).
30. Nguyen et al., Fundamentals and Applications of Microfluidics, Second Edition, ARTECH HOUSE, INC. (Norwood, Mass.: 2006), pages 395-417
31. Li et al., Injection Schemes for Microchip-based Analysis Systems, Lab on a Chip Technology, Volume 1: Fabrication and Microfluidics, edited by Herold et al., Caister Academic Press (Norfolk, UK: 2009), pages 385-403
32. Armani et al., Fabricating PDMS Microfluidic Channels Using a Vinyl Sign Plotter, Lab on a Chip Technology, Volume 1: Fabrication and Microfluidics, edited by Herold et al., Caister Academic Press (Norfolk, UK: 2009), pages 9-15
33. Tsao et al., Bonding Techniques for Thermoplastic Microfluidics, Lab on a Chip Technology, Volume 1: Fabrication and Microfluidics, edited by Herold et al., Caister Academic Press (Norfolk, UK: 2009), pages 45-63.
34. Carlen et al., Silicon and Glass Micromachining, Lab on a Chip Technology, Volume 1: Fabrication and Microfluidics, edited by Herold et al., Caister Academic Press (Norfolk, UK: 2009), pages 83-114.
35. Cheung et al., Microfluidics-based Lithography for Fabrication of Multi-Component Biocompatible Microstructures, Lab on a Chip Technology, Volume 1: Fabrication and Microfluidics, edited by Herold et al., Caister Academic Press (Norfolk, UK: 2009), pages 115-138.
36. Sun et al., Laminated Object Manufacturing (LOM) Technology-Based Multi-Channel Lab-on-a-Chip for Enzymatic and Chemical Analysis, Lab on a Chip Technology, Volume 1: Fabrication and Microfluidics, edited by Herold et al., Caister Academic Press (Norfolk, UK: 2009), pages 161-172.
37. Waddell, Laser Micromachining, Lab on a Chip Technology, Volume 1: Fabrication and Microfluidics, edited by Herold et al., Caister Academic Press (Norfolk, UK: 2009), pages 173-184.
38. Erickson et al., Introduction to Electrokinetic Transport in Microfluidic Systems, Lab on a Chip Technology, Volume 1: Fabrication and Microfluidics, edited by Herold et al., Caister Academic Press (Norfolk, UK: 2009), pages 231-248.
39. Grover et al., Monolithic Membrane Valves and Pumps, Lab on a Chip Technology, Volume 1: Fabrication and Microfluidics, edited by Herold et al., Caister Academic Press (Norfolk, UK: 2009), pages 285-317.
40. Hunt et al., Integrated Circuit/Microfluidic Chips for Dielectric Manipulation, Lab on a Chip Technology, Volume 2: Biomolecular Separation and Analysis, edited by Herold et al., Caister Academic Press (Norfolk, UK: 2009), pages 187-206.
41. Bersano-Begey et al., Braille Microfluidics, Lab on a Chip Technology, Volume 2: Biomolecular Separation and Analysis, edited by Herold et al., Caister Academic Press (Norfolk, UK: 2009), pages 269-285.
42. Petchakup, C. et al. Label-free quantitative lymphocyte activation profiling using microfluidic impedance cytometry. Sensors and Actuators B: Chemical 339, 129864, doi:https://doi.org/10.1016/i.snb.2021.129864 (2021)
43. De Ninno, A. et al. Coplanar electrode microfluidic chip enabling accurate sheathless impedance cytometry. Lab on a Chip, doi:10.1039/C6LC01516F (2017).
44. Spencer, D. & Morgan, H. Positional dependence of particles in microfludic impedance cytometry. Lab on a Chip 11, 1234-1239, doi:10.1039/C1 LC20016J (2011).
45. Armistead, F. J., Gala De Pablo, J., Gadélha, H., Peyman, S. A. & Evans, S. D. Cells Under Stress: An Inertial-Shear Microfluidic Determination of Cell Behavior. Biophys J 116, 1127-1135, (2019).
46. Armistead, F. J., Gala De Pablo, J., Gad6lha, H., Peyman, S. A. & Evans, S. D. Cells Under Stress: An Inertial-Shear Microfluidic Determination of Cell Behavior. Biophysical Journal 116, 1127-1135, doi:https://doi.org/10.1016/j.bpi.2019.01.034 (2019).
47. Dudani, J. S., Gossett, D. R., Tse, H. T. K. & Di Carlo, D. Pinched-flow hydrodynamic stretching of single-cells. Lab on a Chip 13, 3728-3734, doi:10.1039/C3LC50649E (2013).
48. Petchakup, C., Tay, H. M., Li, K. H. H. & Hou, H. W. Integrated inertial-impedance cytometry for rapid label-free leukocyte isolation and profiling of neutrophil extracellular traps (NETs).

### Lab on a Chip 19, 1736-1746, (2019).

49. Song, H. et al. A microfluidic impedance flow cytometer for identification of differentiation state of stem cells. Lab on a Chip 13, 2300-2310, doi:10.1039/C3LC41321G (2013).
50. Petchakup, C. et al. Label-free leukocyte sorting and impedance-based profiling for diabetes testing. Biosensors and Bioelectronics 118, 195-203.
51. Otto, O. et al. Real-time deformability cytometry: on-the-fly cell mechanical phenotyping. Nat Methods 12, 199-202, (2015).
52. Kim, S.-O., Kim, J., Okajima, T. & Cho, N.-J. Mechanical properties of paraformaldehyde-treated individual cells investigated by atomic force microscopy and scanning ion conductance microscopy. Nano Convergence 4, 5, (2017).
53. Ekpenyong, A. E. et al. Viscoelastic Properties of Differentiating Blood Cells Are Fate- and Function-Dependent. PLOS ONE 7, e45237, doi:10.1371/journal.pone.0045237 (2012).
54. Ekpenyong, A. E. et al. Mechanical deformation induces depolarization of neutrophils. Sci Adv 3, el602536-e1602536, doi:10.1126/sciadv.1602536 (2017).
55. van Oostrom, A. J., van Wijk, J. P., Sijmonsma, T. P., Rabelink, T. J. & Castro Cabezas, M.

Increased expression of activation markers on monocytes and neutrophils in type 2 diabetes.

The Netherlands journal of medicine 62, 320-325 (2004).
56. Zhang, Y., Chen, X., Gueydan, C. & Han, J. Plasma membrane changes during programmed cell deaths. Cell Research 28, 9-21, (2018).
57. Toepfner, N. et al. Detection of human disease conditions by single-cell morpho-rheological phenotyping of blood. eLife 7, e29213, (2018).
58. Vlassakis, J. et al. Measuring expression heterogeneity of single-cell cytoskeletal protein complexes. Nature Communications 12, 4969, (2021).
59. Iba, T., Hashiguchi, N., Nagaoka, I., Tabe, Y. & Murai, M. Neutrophil cell death in response to infection and its relation to coagulation. Journal of Intensive Care 1, 13, (2013).
60. Kenny, E. F. et al. Diverse stimuli engage different neutrophil extracellular trap pathways. eLife 6, (2017).
61. Tse, W. Y., Nash, G. B., Hewins, P., Savage, C. O. & Adu, D. ANCA-induced neutrophil F-actin polymerization: implications for microvascular inflammation. Kidney international 67, 130-139, (2005).
62. Neubert, E. et al. Chromatin swelling drives neutrophil extracellular trap release. Nature Communications 9, 3767, (2018).
63. Xavier, M., de Andrés, M. C., Spencer, D., Oreffo, R. O. C. & Morgan, H. Size and dielectric properties of skeletal stem cells change critically after enrichment and expansion from human bone marrow: consequences for microfluidic cell sorting. Journal of The Royal Society Interface 14, 20170233, (2017).
64. Zeming, K. K. et al. Label-Free Biophysical Markers from Whole Blood Microfluidic Immune Profiling Reveal Severe Immune Response Signatures. Small 17, 2006123, (2021).
65. Kubdnkovd, M. et al. Physical phenotype of blood cells is altered in COVID-19.

1. An optics-free method for single-cell profiling, comprising:
(i) forming a single stream of cells in a sample flow channel of a microfluidic device by perfusing a fluid sample containing the cells through a particle focusing region of the microfluidic device, wherein the particle focusing region is upstream of a detection region of the microfluidic device, wherein the sample flow channel extends through the particle focusing region and the detection region;
(ii) measuring an impedance response of each cell by a first pair of electrodes disposed along the sample flow channel within the detection region;
(iii) deforming each cell within a cell deformation zone along the sample flow channel within the detection region, wherein the cell deformation zone is downstream of the first pair of electrodes;
(iv) measuring an impedance response of each cell after deformation by a second pair of electrodes disposed along the sample flow channel within the detection region of the microfluidic device, wherein the second pair of electrodes is downstream of the cell deformation zone;
(v) determining one or more quantitative relationships among impedance responses from one or a combination of the first and second pair of electrodes to quantify one or more biophysical properties of each cell for profiling of said cell in said fluid sample.

2. The method of claim 1, wherein the cells are blood cells, preferably neutrophils.

3. The method of claim 1, wherein the one or more biophysical properties comprise:
(a) membrane opacity;
(b) nucleus opacity;
(c) cell size; and
(d) deformability index.

4. The method of claim 1, wherein measuring the impedance response by each pair of the first and second pair of electrodes comprises measuring a multi-frequency impedance response, wherein a multi-frequency excitation signal is applied by each pair of the first and second pair of electrodes for measuring the multi-frequency impedance response.

5. The method of claim 4, wherein the multi-frequency impedance response comprises impedance signals at three or more frequencies, wherein the three or more frequencies are in a range of about 0.1 MHz to 20 MHz, wherein the impedance signals at the three or more frequencies comprise a first impedance signal at a first frequency in a range of about 0.1 MHz to 0.5 MHz, a second impedance signal at a second frequency in a range of about 1 MHz to 5 MHz, and a third impedance signal at a third frequency in a range of about 5 MHz to 20 MHz, preferably the impedance signals at the three or more frequencies comprise the first impedance signal at a first frequency of about 0.3 MHz, the second impedance signal at a second frequency of about 1.7 MHz, and the third impedance signal at a third frequency of about 12 MHz.

6.-7. (canceled)

8. The method of claim 5, wherein determining one or more quantitative relationships among impedance responses from the first and/or second pair of electrodes comprises:
(i) determining a ratio of the second impedance signal at the second frequency and the first impedance signal at the first frequency to quantify a membrane opacity of each cell as one of the one or more biophysical properties for said cell; and/or
(ii) determining a ratio of the third impedance signal at the third frequency and the first impedance signal at the first frequency to quantify a nucleus opacity of each cell as one of the one or more biophysical properties for said cell; and/or
(iii) determining a ratio of the impedance response of the first pair of electrodes and the impedance response of the second pair of electrodes to quantify a deformability index of each cell as one of the one or more biophysical properties for said cell.

9. (canceled)

10. The method of claim 5, further comprising determining the first impedance signal at the first frequency of the first pair of electrodes to quantify a cell size of each cell.

11. (canceled)

12. The method of claim 1, wherein deforming each cell within the cell deformation zone comprises hydrodynamically deforming said cell by perfusing a sheath fluid along two split flow paths to converge in an intersecting manner with the sample flow channel at the cell deformation zone from two opposite sides of the sample flow channel so as to apply a hydrodynamic force for deforming each cell flowing through the cell deformation zone along the sample flow channel, optionally wherein the two split flow paths intersect the sample flow channel to form a cross-junction.

13. (canceled)

14. The method of claim 12, wherein perfusing the fluid sample is at a sample flow rate and perfusing the sheath fluid is at a sheath flow rate, wherein the sheath flow rate is higher than the sample flow rate, optionally wherein the sample flow rate is in the range of 1 to 20 μL/min, and the sheath flow rate is in the range of 1 to 40 μL/min.

15. (canceled)

16. The method of claim 1, wherein the fluid sample comprises a viscoelastic medium, preferably poly(ethylene oxide), in phosphate-buffered saline, and the sheath fluid consists of a viscoelastic medium, preferably poly(ethylene oxide).

17. The method of claim 16, wherein forming the single stream of cells in the sample flow channel comprises aligning the cells into the single stream of cells along a center of the sample flow channel by viscoelastic focusing effect, optionally, wherein aligning the cells into the single stream of cells along the center of the sample flow channel by viscoelastic focusing effect comprises perfusing the fluid sample containing the cells through a winding path section of the sample flow channel forming the particle focusing region of the microfluidic device, wherein the winding path section of the sample flow channel has a pre-determined length based on a viscoelasticity of the viscoelastic medium in order to complete alignment of the cells into the single stream of cells by viscoelastic focusing effect within the winding path section.

18. (canceled)

19. An optics-free system for single-cell profiling, the system comprises:
a microfluidic device comprising a sample flow channel extending through a particle focusing region and a detection region, wherein the detection region comprises a cell deformation zone, a first pair of electrodes and a second pair of electrodes, wherein the first and second pair of electrodes are respectively arranged before and after the cell deformation zone; and
a computing system comprising: a memory; and at least one processor communicatively coupled to the memory and the first and second pair of electrodes of the microfluidic device,
wherein the computing system is configured to:
(i) measure an impedance response of each cell flowing past the first pair of electrodes disposed along the sample flow channel within the detection region;
(ii) measure an impedance response of each cell after deformation flowing past the second pair of electrodes disposed along the sample flow channel within the detection region; and
(iii) determine one or more quantitative relationships among impedance responses from one or a combination of the first and second pair of electrodes to quantify one or more biophysical properties of each cell for profiling of said cell.

20. The system of claim 19, wherein the computing system is configured to determine a ratio of the impedance response of the first pair of electrodes and the impedance response of the second pair of electrodes to quantify a deformability index of each cell as one of the one or more biophysical properties for said cell.

21. The system of claim 19, further comprising a lock-in amplifier and one or more transimpedance amplifiers, wherein the lock-in amplifier communicatively coupled between the computing system and the first and second pair of electrodes of the microfluidic device, wherein each pair of the first and second pair of electrodes is configured to apply a multi-frequency excitation signal to each cell, wherein the computing system is configured to measure, via the lock-in amplifier, a multi-frequency impedance response by each pair of the first and second pair of electrodes, wherein the one or more transimpedance amplifiers is connected to one electrode of each pair to convert impedance responses for the lock-in amplifier.

22. The system of claim 21, wherein the computing system is configured to:
measure, via the lock-in amplifier, the multi-frequency impedance response comprising impedance signals at three or more frequencies, wherein the three or more frequencies are in a range of about 0.1 MHz to 20 MHz, wherein the impedance signals at the three or more frequencies comprise a first impedance signal at a first frequency in a range of about 0.1 MHz to 0.5 MHz, a second impedance signal at a second frequency in a range of about 1 MHz to 5 MHz, and a third impedance signal at a third frequency in a range of about 5 MHz to 20 MHz, preferably, the impedance signals at the three or more frequencies comprise the first impedance signal at a first frequency of about 0.3 MHz, the second impedance signal at a second frequency of about 1.7 MHz, and the third impedance signal at a third frequency of about 12 MHz.

23.-24. (canceled)

25. The system of claim 23, wherein the computing device is configured to:
(i) determine a ratio of the first impedance signal at the first frequency and the second impedance signal at the second frequency to quantify a membrane opacity of each cell as one of the one or more biophysical properties for said cell; and/or
(ii) determine a ratio of the first impedance signal at the first frequency and the third impedance signal at the third frequency to quantify a nucleus opacity of each cell as one of the one or more biophysical properties for said cell; and/or
(iii) quantify a cell size of each cell based on the first impedance signal at the first frequency of the first pair of electrodes.

26.-27. (canceled)

28. A microfluidic device for single-cell profiling, comprising:
a first flow channel to form a fluid pathway for allowing a fluid sample comprising cells to flow from a sample inlet to an outlet, wherein the first flow channel comprises a particle focusing region;
a second flow channel to form a fluid pathway for allowing a sheath fluid to flow from a sheath fluid inlet to a junction, wherein the second flow channel intersects with the first flow channel to form the junction in a cell deformation zone;
two pairs of electrodes arranged adjacent to the junction, wherein the at least two pairs of electrodes and the junction define a detection region,
wherein the two pairs of electrodes comprise a first pair of electrodes and a second pair of electrodes, and
wherein the first pair of electrodes extend across the first flow channel upstream of the junction, and wherein the second pair of electrodes extend across the first flow channel downstream of the junction.

29. The microfluidic device of claim 28, wherein the second flow channel is bifurcated into two split flow pathways that converge in an intersecting manner with the first flow channel to form the junction, wherein the junction is a cross-junction.

30. The microfluidic device of claim 28, wherein the particle focusing region is upstream of the detection region, wherein the first flow channel extends through the particle focusing region, the cell deformation zone and the detection region.

31. The microfluidic device of claim 28, wherein the detection region comprises the cell deformation zone along the first flow channel, wherein the cell deformation zone is downstream of the first pair of electrodes, optionally, wherein the second pair of electrodes is downstream of the cell deformation zone.

32.-33. (canceled)

34. The microfluidic device of claim 28, wherein the first pair of electrodes are substantially parallel to each other, and the second pair of electrodes are substantially parallel to each other.
